(19) United States
(12) Patent Application Publication
(10) Pub. No.: US 2020/0147214 A1
(43) Pub. Date: May 14, 2020

(54) TISSUE FILLER COMPOSITIONS FOR PHOTO-BIOMODULATION AND METHODS FOR ACHIEVING SAME

(71) Applicant: KLOX TECHNOLOGIES INC., Laval (CA)

(72) Inventors: Andreas NIKOLIS, Ville Mont-Royal (CA); Nikolaos LOUPIS, Athens (GR); Remigio PIERGALLINI, San Benedetto del Tronto (IT)

(21) Appl. No.: 16/632,459

(22) PCT Filed: Jul. 18, 2018
(86) PCT No.: PCT/CA2018/050872

§ 371 Date: Jan. 20, 2020

Related U.S. Application Data
(60) Provisional application No. 62/534,447, filed on Jul. 19, 2017.
(60) Provisional application No. 62/534,612, filed on Jul. 19, 2017.
(60) Provisional application No. 62/696,523, filed on Jul. 11, 2018.

(51) Int. Cl. A61K 41/00; A61K 47/36; A61L 27/20; A61N 5/06; A61K 31/352

(52) U.S. Cl. CPC A61K 41/00; A61K 47/36; A61L 27/20; A61N 5/062; A61L 2400/06; A61N 2005/0651; A61N 2005/0663; A61L 2300/216; A61K 31/352

(57) ABSTRACT

The present disclosure generally relates to photo-biomodulation composition comprising a tissue filler and to methods for achieving photo-biomodulation. Specifically, but not exclusively, the present disclosure relates to photo-biomodulation compositions which can be injected into a tissue for achieving photo-biomodulation.

### CROSS-REFERENCE TO RELATED APPLICATIONS

This application claims the benefit of and priority to U.S. provisional patent application No. 62/534,447, filed on Jul. 18, 2017; to U.S. provisional patent application No. 62/534,612, filed on Jul. 18, 2017; and to U.S. provisional patent application No. 62/696,532, filed on Jul. 11, 2018, the content of all of which is herein incorporated in entirety by reference.

FIELD OF TECHNOLOGY

The present disclosure generally relates to compositions and methods for achieving photo-biomodulation. Specifically, but not exclusively, the present disclosure relates to compositions comprising tissue filler which may be injected or implanted into tissues for achieving photo-biomodulation.

### BACKGROUND INFORMATION

In recent years, tissue fillers such as hyaluronic acid (HA)-based fillers have become the material of choice for use in soft tissue and dermal correction, for the most part replacing collagen fillers such as Zyderm®, Zyplast®, Volbella®, Voluma®, Cosmoderm®, Juvederm®, and Cosmoplast® (Allergan, Irvine, Calif., USA), Hylaform®, Captique®, and Prevelle® (Genzyme Co., Cambridge, Mass., USA), Restylane® (Q-Med, Uppsala, Sweden), Belotero® (Anteis SA, Geneva, Switzerland), Puragen® (Mentor, Santa Barbara, Calif., USA), Perlan® and Emervel® (Galderma, Lausanne, Switzerland).

Although tissue fillers appear to be similar, their physical characteristics and methods of manufacture may be different. These differences have clinical ramifications for the physician in that they can affect injection technique, usage, and the quality of the outcome.

The molecular weight of HA is proportional to the number of repeating disaccharides in the HA molecule. The HA used in manufacturing dermal fillers can range from 500 to 6000 kDa. Commercial preparations of HA are usually supplied as corresponding sodium salt and have a disaccharide molecular weight of approximately 400 Da. High molecular weight HA has a molecular weight of at least about 1.0 million Daltons (1×10⁶ Da or 1 MDa) to about 4.0 MDa, whereas low molecular weight HA has a molecular weight of less than about 1.0 MDa.

In its natural state, HA exhibits poor biochemical properties as tissue filler. HA has excellent biocompatibility and affinity for water molecules but it is a soluble polymer that is cleared rapidly when injected into normal skin. Therefore, to provide the ability to lift and fill wrinkles in the skin, chemical modifications are required to improve its mechanical properties. Cross-linking has been used to improve biochemical properties of HA while maintaining its biocompatibility and biological activity. The most common cross-linkers for dermal fillers are divinyl sulfone (Hylaform®, Captique®, and Prevelle®, Genzyme Co., Cambridge, Mass., USA) and diglycidyl esthers (Restylane®, Q-Med®, Uppsala, Sweden; Juvederm®, Allergan, Irvine, Calif., USA; and Belotero®, Anteis SA, Geneva, Switzerland) or bis-epoxides (Puragen®, Mentor, Santa Barbara, Calif., USA). Other examples of cross-linkers include: 1,4-butanediol diglycidyl ether (BDDE), 1,4-bis(2,3-epoxypropoxy)butane, 1,4-bisglycidyloxybutane, 1,2-bis(2,3-epoxypropoxy)ethylene and 1-(2,3-epoxypropyl)-2,3-epoxycyclohexane, and 1,4-butanediol diglycidyl ether.

As the cross-link density of a gel increases, the distance between the cross-linked segments become shorter. Increasing cross-link density strengthens the overall network, thereby increasing the hardness or stiffness of the gel. However, when the gel comprises all or mostly pendant HA modification, a low cross-link-density network is formed resulting in softer gels.

In addition to providing tissue filler properties for a sustained period of time, it would be of interest that tissue fillers and in particular HA-based tissue fillers be developed so as to be capable of performing other beneficial effects to the skin at and around the site of injection. In view of this, the present disclosure provides new and improved tissue filler compositions that exhibit photo-biomodulation while being retained at the site of injection.

### SUMMARY OF DISCLOSURE

According to various aspects, the present disclosure relates to a method for effecting photo-biomodulation in a tissue, the method comprising: administering at least one tissue filler to the tissue; and illuminating the tissue with a source of actinic light; wherein the illumination of the tissue filler with actinic light effects biomodulation in the tissue.

According to various aspects, the present disclosure relates to the use of at least one tissue filler to effect photo-biomodulation of a tissue, wherein illumination of the tissue filler with a source of actinic light effects biomodulation in the tissue.

According to various aspects, the at least one tissue filler is administered in combination with at least one light-absorbing molecule. In some instances, the light-absorbing molecule is a xanthene dye, such as, but not limited to Eosin B, Eosin Y, Eosin derivative, Erythrosine, Fluorescein, Phloxin B and Rose Bengal. In some instances, the light-absorbing molecule is Eosin Y and Fluorescein. In some other instances, the light-absorbing molecule is dispersed between chains of the tissue filler.

According to various aspects, the present disclosure relates to a method for effecting photo-biomodulation in a soft tissue, the method comprising: administering a photo-biomodulation composition to a tissue, wherein the photobiomodulation composition comprises at least one light-absorbing molecule and at least one tissue filler; and illuminating the tissue with a source of actinic light; wherein the illumination of the tissue filler with actinic light effects biomodulation in the soft tissue.

According to various aspects, the present disclosure relates to the use of at least one photo-biomodulation composition to effect photo-biomodulation of a tissue, wherein the photo-biomodulation composition comprises at least one light-accepting molecule and at least one tissue filler; and wherein illumination of the photo-biomodulation composition with a source of actinic light effects biomodulation in the tissue.

According to various aspects, the present disclosure relates to a system for effecting photo-biomodulation in a tissue, the system comprising: a photo-biomodulation composition in a form suitable for administration into the tissue; wherein the photo-biomodulation composition comprises at least one light-absorbing molecule and at least one tissue filler; and a source of actinic light; wherein illumination of the photo-biomodulation composition injected into the tissue with actinic light emitted from the source of actinic light triggers biomodulation of the tissue by the tissue filler.

According to various aspects, the present disclosure relates to the use of a photo-biomodulation composition comprising at least one tissue filler and at least one light-absorbing molecule in a method for modulating collagen synthesis in a tissue, wherein the method comprises a step of administration of the photo-biomodulation composition to an area to be treated within the tissue, and a step illumination of the area with light having a wavelength which can stimulate the at least one tissue filler and which can be absorbed by the at least one light-absorbing molecule; and wherein the method biomodulates collagen synthesis in the area.

According to various aspects, the present disclosure relates to the use of a photo-biomodulation composition comprising at least one tissue filler and at least one light-absorbing molecule in a method for management of a wound, wherein the method comprises a step of administration of the photo-biomodulation composition to an area of skin comprising the wound, and a step illumination of the area with light having a wavelength which can stimulate the at least one tissue filler and which can be absorbed by the at least one light-absorbing molecule; and wherein the method biomodulates collagen synthesis in the area.

According to various aspects, the present disclosure relates to a tissue filler composition comprising: at least one tissue filler; and at least one light-absorbing molecule; wherein the composition is substantially colorless and is suitable for injection into a tissue.

According to various aspects, the present disclosure relates to a combination comprising at least one tissue filler in a form suitable for administration into a tissue, and a source of actinic light, the source of actinic light for stimulating the at least one tissue filler, wherein stimulation of the at least one tissue filler by actinic light emitted by the source of actinic light triggers biomodulation in the tissue.

According to various aspects, the present disclosure relates to a combination comprising at least one tissue filler in a form suitable for administration into a tissue, at least one light-absorbing molecule, and a source of actinic light, the source of actinic light for stimulating the at least one tissue filler and activating the at least one light-absorbing molecule, wherein stimulation of the at least one tissue filler and activation of the at least one light-absorbing molecule by actinic light emitted by the source of actinic light triggers biomodulation in the tissue.

According to various aspects, the present disclosure relates to a combination comprising at least one tissue filler in a form suitable for administration into a tissue, and at least one light-absorbing molecule in a form suitable for administration in to a tissue, wherein stimulation of the at least one tissue filler and activation of the at least one light-absorbing molecule by light triggers biomodulation in the tissue.

According to various aspects, the present disclosure relates to a method for minimizing appearance of fine lines on skin, wherein the method comprises: administering at least one tissue filler into the fine lines; and illuminating the fine lines with a source of actinic light; wherein the illumination of the fine lines with actinic light minimizes the appearances of the fine lines.

According to various aspects, the present disclosure relates to a method for minimizing appearance of fine lines on skin, wherein the method comprises: administering at least one tissue filler and at least one light-absorbing molecule into the fine lines; and illuminating the fine lines with a source of actinic light; wherein the illumination of the fine lines with actinic light minimizes the appearances of the fine lines.

According to various aspects, the present disclosure relates to a tissue filler composition, wherein the tissue filler composition is a cross-linked tissue filler network capable of retaining and transferring energy upon being stimulated by actinic light emitted by a light source.

According to various aspects, the present disclosure relates to a tissue filler composition, wherein the tissue filler composition is a cross-linked tissue filler network capable of retaining and transferring energy to one or more light-absorbing molecules upon being stimulated by actinic light emitted by a light source.

According to various aspects, the present disclosure relates to a method for effecting photo-biomodulation in a tissue, the method comprising: administering a first photo-biomodulation composition into an area of a tissue, wherein the photo-biomodulation composition comprises at least one light-absorbing molecule and at least one tissue filler; topically applying a second photo-biomodulation composition to the area of the tissue; and illuminating the area of the tissue with a source of actinic light; wherein the illumination of the area of the tissue with actinic light effects biomodulation in the tissue.

According to various aspects, the light-absorbing molecules of the present technology are cross-linked to the components of the tissue filler; more particularly, the light-absorbing molecules are cross-linked to the components of the tissue filler network.

### BRIEF DESCRIPTION OF THE DRAWINGS

FIG. 1 is schematic representation of an injection program in pig models according to one embodiment of the present technology.

FIGS. 2A-2I are graphs illustrating the effect of subcutaneous co-injection of Emervel® Classic (EC) as tissue filler together with a photo-biomodulation composition on Decorin expression (FIGS. 2A, 2B, 2C), on Collagen III expression (FIGS. 2D, 2E, 2F) and on Ki67 expression (FIGS. 2G, 2H, 2I) in the injected skin of the pig model at 2 months, 4 months and 6 months post photo-treatment.

FIGS. 3A-3I are graphs illustrating the effect of subcutaneous co-injection of Emervel® Volume (EV) as tissue filler together with a photo-biomodulation composition on Decorin expression (FIGS. 3A, 3B, 3C), on Collagen III expression (FIGS. 3D, 3E, 3F) and on Ki67 expression (FIGS. 3G, 3H, 3I) in the injected skin of the pig model at 2 months, 4 months and 6 months post photo-treatment.

FIGS. 4A-4I are graphs illustrating the effect of subcutaneous co-injection of Perlane® (Per L) as tissue filler together with a photo-biomodulation composition on Decorin expression (FIGS. 4A, 4B, 4C), on Collagen III expression (FIGS. 4D, 4E, 4F) and on Ki67 expression (FIGS. 4G, 4H, 4I) in the injected skin of the pig model at 2 months, 4 months and 6 months post photo-treatment.

FIGS. 5A-5I are graphs illustrating the effect of subcutaneous co-injection of Radiesse® (Rad) as tissue filler together with a photo-biomodulation composition on Decorin expression (FIGS. 5A, 5B, 5C), on Collagen III expression (FIGS. 5D, 5E, 5F) and on Ki67 expression (FIGS. 5G, 5H, 5I) in the injected skin of the pig model at 2 months, 4 months and 6 months post photo-treatment.

FIGS. 6A-6I are graphs illustrating the effect of subcutaneous co-injection of Restylane® (RL) as tissue filler together with a photo-biomodulation composition on Decorin expression (FIGS. 6A, 6B, 6C), on Collagen III expression (FIGS. 6D, 6E, 6F) and on Ki67 expression (FIGS. 6G, 6H, 6I) in the injected skin of the pig model at 2 months, 4 months and 6 months post photo-treatment.

FIGS. 7A-7I are graphs illustrating the effect of subcutaneous co-injection of Restylane® Fine Line (RFL) as tissue filler together with a photo-biomodulation composition on Decorin expression (FIGS. 7A, 7B, 7C), on Collagen III expression (FIGS. 7D, 7E, 7F) and on Ki67 expression (FIGS. 7G, 7H, 7I) in the injected skin of the pig model at 2 months, 4 months and 6 months post photo-treatment.

FIG. 8 shows graphs illustrating the effect of the photo-biomodulation compositions according to the present technology on Decorin expression (Panel A), Collagen III expression (Panel B), and Ki67 expression (Panel C) in the injected skin of the pig model at 2 months, 4 months and 6 months post photo-treatment. Filler=dermal filler, PL LAM=absence of light-absorbing molecules; PL LED=absence of light (dark room); LAM 1=Eosin Y; LAM 2=Eosin Y+Fluorescein; B LED=Blue LED; G LED=Green LED.

FIGS. 9A-9B are graphs illustrating the effect of the effect of subcutaneous co-injection of Emervel® Classic (EC) as tissue filler together with a photo-biomodulation composition on Decorin expression (FIG. 9A) and on Collagen III expression (FIG. 9B) in the injected skin of the pig model at 2 months, 4 months and 6 months post photo-treatment.

FIGS. 9C-9D are graphs illustrating the effect of the effect of subcutaneous co-injection of EmerVel® Volume (EV) as tissue filler together with a photo-biomodulation composition on Decorin expression (FIG. 9C) and on Collagen III expression (FIG. 9D) in the injected skin of the pig model at 2 months, 4 months and 6 months post photo-treatment.

FIGS. 9E-9F are graphs illustrating the effect of the effect of subcutaneous co-injection of Radiance® (Rad) as tissue filler together with a photo-biomodulation composition on Decorin expression (FIG. 9E) and on Collagen III expression (FIG. 9F) in the injected skin of the pig model at 2 months, 4 months and 6 months post photo-treatment.

FIGS. 9G-9H are graphs illustrating the effect of the effect of subcutaneous co-injection of Restylane® Fine Line (RFL) as tissue filler together with a photo-biomodulation composition on Decorin expression (FIG. 9G) and on Collagen III expression (FIG. 9H) in the injected skin of the pig model at 2 months, 4 months and 6 months post photo-treatment.

FIGS. 9I-9J are graphs illustrating the effect of the effect of subcutaneous co-injection of Restylane® (RL) as tissue filler on Decorin expression (FIG. 9I) and on Collagen III expression (FIG. 9J) in the injected skin of the pig model at 2 months, 4 months and 6 months post photo-treatment

It is to be expressly understood that the description and drawings are only for the purpose of illustrating certain embodiments of the present technology and are an aid for understanding. They are not intended to be a definition of the limits of the technology.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant technology. Hence, the following specification is intended to illustrate some particular embodiments of the technology, and not to exhaustively specify all permutations, combinations and variations thereof.

As used herein, the singular form “a,” “an” and “the” include plural referents unless the context clearly dictates otherwise.

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “biophotonic” means the generation, manipulation, detection and application of photons in a biologically relevant context. In other words, biophotonic compositions exert their physiological effects primarily due to the generation and manipulation of photons. As used herein, the expression “biophotonic composition” refers to a composition as described herein that may be activated by light to produce photons for biologically relevant applications. The biophotonic system of the present technology comprises an LED light and a light-absorbing molecule (light-trapping acceptor)-containing gel.

The term “topical” as used herein means as applied to body surfaces, such as the skin, mucous membranes, vagina, oral cavity, internal surgical wound sites, and the like.

As used herein, the term “injection” refers to the act of putting a substance (e.g., liquid), into a person's body using a needle (usually a hypodermic needle) and a syringe. Injection is a technique for delivering a substance by parenteral administration, that is, administration via a route other than through the digestive tract. The injection may be a single injection or may be a series of injections. Parenteral injection includes subcutaneous injection, intramuscular injection, intravenous injection, intraperitoneal injection, intracardiac injection, intraarticular injection and intracavernous injection. In subcutaneous injection, the substance is delivered to the tissues (e.g., subcutaneous tissues) between the skin and the muscle. In an intradermal injection, the substance is delivered directly into the dermis, the layer just below the epidermis of the skin. In some embodiments, subcutaneous injections include injections onto the bone or in proximity of the bone. A depot injection is an injection, usually subcutaneous, intradermal, or intramuscular, that deposits a substance in a localized mass, called a depot, from which it is gradually absorbed by surrounding tissue. Such injection allows the photoactivatable composition to be released in a consistent way over a long period. Depot injections are usually either solid or oil-based.

The term “injectable” when used herein means a flowable material which may be drawn through or pushed through a needle by a syringe for injection into a soft tissue. Soft tissue includes tendons, ligaments, fascia, skin, dermis, fibrous tissues, fat, synovial membranes, muscles, nerves and blood vessels.

As used herein, the term “epidermis” refers to the outer layer of the three layers that make up the skin, the inner layers being the dermis and hypodermis. The epidermis layer provides a barrier to infection from environmental pathogens and regulates the amount of water released from the body into the atmosphere through transepidermal water loss. The outermost part of the epidermis is composed of stratified layers of flattened cells, that overlies a basal layer (stratum basale) composed of columnar cells arranged perpendicularly. As used herein, the term “dermis” refers to the thick layer of living tissue below the epidermis that forms the true skin, containing blood capillaries, nerve endings, sweat glands, hair follicles, and other structures.

The following terms and expressions: “light-absorbing molecule”, “light-accepting molecule”, “photoactivating agent”, “photoactivator” and “chromophore” are used herein interchangeably. A light-absorbing molecule means a molecule or a compound or a complex of molecules, which when contacted by light irradiation, is capable of absorbing the light. The light-absorbing molecule readily undergoes photoexcitation and can then transfer its energy to other molecules or emit it as light.

As used herein, the expression “photo-biomodulation” refers to the use of light to effect modulation of biological and/or biochemical processes.

As used herein, the expression “soft tissue” refers to tissues that connect, support, or surround other structures and organs of the body, not being bone. Soft tissue includes tendons, ligaments, fascia, skin, fibrous tissues, fat, axilla, and synovial membranes (which are connective tissue), and muscles, nerves and blood vessels (which are not connective tissue).

The term “light” or “actinic light” is intended to mean light energy emitted from a specific light source (e.g., lamp, LED, laser) and capable of being absorbed by matter (e.g., the light-absorbing molecule defined above). In a preferred embodiment, the light has a peak wavelength within the visible range of the electromagnetic spectrum, e.g. about 360 nm to about 760 nm.

The expression “tissue filler” when used herein means a material that is suitable for, or generally used for, tissue augmentation, regeneration or re-shaping such as those suitable for use or used in the dermis area such as dermal fillers, or those suitable for use or used in any other soft tissue for example as a scaffold or a delivery material. Tissue fillers typically include biodegradable and non-biodegradable materials, as well as natural and synthetic materials, including hydrogels. Tissue fillers can be administered by any means such as by injection or implantation. Tissue fillers can be administered to any soft tissue site such as subcutaneous, hypodermic and/or intradermal.

The expression “dermal filler” when used herein means a material which can be used in the dermis area, such as below the epidermis and/or above or below the hypodermis, such as for tissue augmentation, regeneration or re-shaping. Dermal fillers can be delivered by hypodermic and/or intradermal injection, or by any other means such as implantation. Some of the features that differentiate the various dermal fillers and particularly hyaluronic acid (HA) fillers are particle size, the type of crosslinking agent used, the degree of crosslinking, the percentage of cross-linked HA, the amount of free (unmodified) HA present, and G′ (elastic modulus). These physical and chemical attributes will influence the clinical characteristics of each filler such as: clinical indication, ease of injection, degree of tissue filling, longevity, clinical appearance, and side effects.

The expression “elastic modulus (G′)” refers to a number that measures an object or substance's resistance to being deformed elastically (i.e., non-permanently) when a stress is applied to it. The elastic modulus of an object is defined as the slope of its stress-strain curve in the elastic deformation region. A stiffer material will have a higher elastic modulus. n the context of dermal filler, the stiffness or G′ of a filler is an important consideration. G′ is a measurement of gel hardness. It is obtained when a gel is placed on a plate. A second plate is placed over the gel and a lateral force is applied. The measurement of resistance to deformation is known as the elastic modulus or the G′. Together with the cohesivity of the product, G′ values could be used to determine the appropriate placement of an HA dermal filler. For example more robust products (higher G′ values and higher cohesivities) should be used in deeper lines, such as nasolabial folds and marionette lines, as well as to lift the lateral brow, to correct the nasal bridge, to give the ear lobe youthful volume, to evert the nipples, and to raise the nasal tip. More fluid products are more suited to be used over large areas such as the cheekbones and cheeks. Low G′ products are necessary in areas that require a softer agent, such as the body of the lip or the tear trough.

The present disclosure stems from the results of experiments conducted in order to assess the influence of various types of tissue fillers on photo-biomodulation by light-absorbing molecules. In particular, the present disclosure stems from the assessment of the influence of different types of chemical modifications (e.g., cross-linking) of tissue fillers on photo-biomodulation by light-absorbing molecules.

The experimental data presented herein indicates that the effects of photo-activated light-absorbing molecules on modulation of biological processes, such as, but not limited to, on collagen synthesis and on wound management, may be modulated when light-absorbing molecules are co-administered with a tissue filler, in particular a cross-linked tissue filler.

The experiment data presented herein thus indicates that different physicochemical properties of tissue fillers, associated with distinct manufacturing technologies, may influence the biomodulation of biological/biochemical processes as a result of photo-activation/photo-stimulation of the light-absorbing molecules.

In addition, the data presented herein further suggests that photo-stimulated cross-linked tissue fillers have the ability to modulate biological processes in absence of light-absorbing molecules.

In on embodiment, the present disclosure provides compositions useful as tissue fillers and as photo-biomodulators. In some implementations of this embodiment, the compositions are photo-biomodulation compositions (“PBM compositions”). In some instances, the PBM compositions comprise at least one tissue filler. In some instances, the PBM compositions comprise at least one tissue filler and at least one light-absorbing molecule. In some other instances, the PBM compositions comprise at least one tissue filler and no light-absorbing molecule. In some other instances, the PBM compositions comprise at least one light-absorbing molecule and no tissue filler.

In one embodiment, the present disclosure relates to a method for photo-biomodulation of a tissue (e.g., soft tissue). The method comprising the step of administering a PBM composition of the present disclosure to an area to be treated within a tissue, wherein the PBM composition comprises at least one tissue filler and at least one light-absorbing molecule; and the step of illuminating the injected area with light having a wavelength which stimulates at least the light-absorbing molecule. Illumination of injected area causes photo-stimulation of the light-absorbing molecule and stimulates biomodulation in the injected area.

In one embodiment, the present disclosure relates to a method for photo-biomodulation of a tissue (e.g., soft tissue). The method comprising the step of administering a PBM composition of the present disclosure to an area to be treated within a tissue, wherein the PBM composition comprises at least one tissue filler and at least one light-absorbing molecule; and the step of illuminating the injected area with light having a wavelength which can stimulate the tissue filler and/or the light-absorbing molecule. Illumination of the injected area causes photo-stimulation of the tissue filler and/or the light-absorbing molecule and stimulates biomodulation in the injected area.

In one embodiment, the present disclosure relates to a method for photo-biomodulation of a tissue (e.g., soft tissue). The method comprising the step of administering a PBM composition of the present disclosure to an area to be treated within a tissue, wherein the PBM composition comprises at least one tissue filler; and the step of illuminating the injected area with light having a wavelength which can stimulate the tissue filler. Illumination of the injected area causes photo-stimulation of the tissue filler and stimulates biomodulation in the injected area.

In one embodiment, the present disclosure relates to a method for photo-biomodulation of a tissue (e.g., soft tissue). The method comprising the step of administering a PBM composition of the present disclosure to an area to be treated within a tissue, wherein the PBM composition comprises at least one tissue filler and at least one light-absorbing molecule; and the step of illuminating the area with light having a wavelength which stimulates the tissue filler and the light-absorbing molecule. Illumination of area, illuminates the tissue filler and the light-absorbing molecule and stimulates biomodulation in the area by the tissue filler.

By means of certain embodiments of the present disclosure, light emitted by the tissue filler and/or by the light-absorbing molecule of the PBM composition can induce or stimulate local biomodulation. In some instances, the local biomodulation is collagen synthesis in the soft tissue around the PBM composition where there otherwise would be no collagen synthesis or where the collagen synthesis would be minimal

In some embodiments, light emitted by the tissue filler and/or by the light-absorbing molecule of the PBM composition can induce or stimulate local biomodulation. In some instances, the local biomodulation allows for management of a wound, such as for example, for treatment of a wound.

The area to be treated may be on any part of the body such as on a face, neck, ears, breasts, buttocks, arms, armpits, hands, genitalia, groin, area between the nose and the upper lip, legs or feet of a human subject. In certain embodiments, the area to be treated is a wound. In certain other embodiments, the ear to be treated is a scar. The scar can be a post-surgical scar. The scar can be an old or a fresh scar. In certain embodiments, the area to be treated is in or around a wound. The wound may be an acute or a chronic wound, including burns. In certain embodiments, the area to be treated includes stretch marks. The area to be treated may be in or around a stretch mark.

In certain embodiments, the administering of the PBM composition is by injection. The injection can be performed using a needle. The needle can have a gauge of 27G to 40G, typically 30G or 32G. The administering by injection can be a continuous injection, also known as linear threading, or serial punctures to deposit microdroplets. In certain other embodiments, the administering of the PBM composition is by implantation.

In certain embodiments, the injection is a subcutaneous injection. In some implementations of this embodiment, the PBM composition is injected to the tissues between the skin and the muscle (e.g., subcutaneous tissues). In some other implementations of this embodiment, the PBM composition is injected into the dermis. In some other implementations, the PBM composition is injected onto the bone or in proximity of the bone. In some implementations, the injection is a single injection wherein the PBM composition is injected at a single location in the soft tissue. In some other implementations, the PBM composition is injected in a plurality of locations in the soft tissue.

In certain other embodiments, the PBM composition is substantially colorless to the human eye. In some other implementations of these embodiments, the PBM composition comprises one or more tissue fillers and the one or more tissue fillers do not confer a color to the PBM composition, that is to say that the one or more tissue fillers are present in the PBM composition in an amount that does not confer color to the PBM composition. In some implementations of these embodiments, the PBM composition comprises one or more light-absorbing molecules and the one or more light-absorbing molecules do not confer a color to the PBM composition, that is to say that the one or more light-absorbing molecules are present in the PBM composition in an amount that does not confer color to the PBM composition. In some other implementations of these embodiments, the PBM composition comprises one or more tissue fillers and one or more light-absorbing molecules and the one or more tissue fillers as well as the one or more light-absorbing molecules do not confer a color to the PBM composition, that is to say that the one or more tissue fillers and the one or more light-absorbing molecules are present in the PBM composition in an amount that does not confer color to the PBM composition.

### i) Tissue Filler

In certain embodiments, the tissue filler comprises a polymer. The polymer may be selected from: proteins, peptides, polypeptides, polylysine, collagens, pro-collagens, elastins, and laminins.

The polymer may also be selected from: synthetic polymers with hydroxyl, amine, and carboxyl functional groups: poly(vinyl alcohol), polyethylene glycol, polyvinlyl amine, polyallylamine, deacetylated polyacrylamide, polyacrylic acid, and polymethacrylic acid. The polymer may also be selected from: dendritic or branched polymers, including dendritic polyols and dendritic polyamines The polymer may further be selected from: solid surface with hydroxyl, amine, and carboxyl functional groups. The polymer may be a polysaccharide, for example, selected from polysaccharides including starch and its derivatives; dextran and its derivatives, cellulose and its derivatives; chitin and chitosan and alginate and its derivatives. In some embodiments, the tissue filler comprises a cross-linked biocompatible polysaccharide gel.

In some embodiments, the polymer is a glycosaminoglycan. The tissue filler medium can further comprise two or more different glycosaminoglycan polymers. As used herein, the term “glycosaminoglycan” is synonymous with “GAG” and “mucopolysaccharide” and refers to long unbranched polysaccharides consisting of a repeating disaccharide units. The repeating unit consists of a hexose (six-carbon sugar) or a hexuronic acid, linked to a hexosamine (six-carbon sugar containing nitrogen) and pharmaceutically acceptable salts thereof. Members of the GAG family vary in the type of hexosamine, hexose or hexuronic acid unit they contain, such as, e.g., glucuronic acid, iduronic acid, galactose, galactosamine, glucosamine) and may also vary in the geometry of the glycosidic linkage. Non-limiting examples of glycosaminoglycans include chondroitin sulfate, dermatan sulfate, keratan sulfate, and hyaluronan. Non-limiting examples of an acceptable salt of a glycosaminoglycans includes sodium salts, potassium salts, magnesium salts, calcium salts, and combinations thereof. Examples of GAGs include, but are not limited to: hyaluronan-based dermal fillers Juvederm®, Juvederm® 30, Juvederm® Ultra, Juvederm® Ultra Plus, Juvederm® Ultra XC, Juvederm™ Ultra Plus XC, Juvederm Voluma® XC, and Juvederm Voluma® (Allergan Inc., Irvine, Calif., USA.).

Examples of biologic, biodegradable tissue filler are those that include materials derived from organism, human, and/or animal tissues and/or products. Examples of such media include the following: hyaluronic acid (HA), (such as the following: avian HA, bovine HA, and non-animal stabilized HA (“NASHA”) (e.g., Restylane®, Captique® and Juvederm® injectable dermal fillers)), collagen (such as collagen I, collagen II, collagen III, cross-linked and/or non-cross-linked, bovine, porcine, human, and autologous collagen). Additional examples of collagen based fillers include Zyplast® (collagen derived from bovine tissue), Zyderm® I (collagen derived from bovine tissue), Zyderm® II, (collagen derived from bovine tissue), Evolence® (porcine derived collagen), and Fibrel® (porcine derived collagen). Collagen-based tissue fillers are generally animal derived and have been associated with a higher occurrence of allergic reactions than non-animal based fillers.

Synthetic, biodegradable, tissue filler media include Radiance® and Radiesse® (calcium and phosphate based microspheres), Laresse® (carboxymethyl cellulose and polyethylene oxide), Sculptra® (microspheres of poly-L-lactic acid), other polyacids, polyethers and polymers. The calcium and phosphate based microspheres comprise calcium hydroxylapatite particles suspended in a water-based gel that acts as a scaffold for new collagen growth.

Other examples of tissue fillers include: Juvederm® Volift from Allergan, Emervel® from Galderma, Elevess® from Anika Therapeutics, Regenovue® from Neogenesis, Restylane® (Q-Med AB, Uppsala, Sweden), Restylane® Fine Line (Q-Med AB, Uppsala, Sweden).

In some embodiments, the tissue filler is hyaluronic acid (HA). In some instances, the HA is cross-linked HA. The cross-linked hyaluronic acid may be about 0.1% to about 2%, about 2% to about 30%, about 2% to about 25%, about 2% to about 20%, about 2% to about 15%, about 2% to about 10%, about 2% to about 5%, 4% to about 30%, about 4% to about 25%, about 4% to about 20%, about 4% to about 15%, about 4% to about 10%, about 4% to about 5%, of the PBM composition. In some other embodiments, the cross-linked HA is Perlane/Restylane® (Galderma, Lausanne, Switzerland), Emervel® (Galderma, Lausanne, Switzerland), Volbella® (Allergan Inc., Irvine, Calif., USA), Voluma® (Allergan Inc., Irvine, Calif., USA), Radiesse® (Merz Aesthetics, NC, USA) or Restylane (Q-Med AB, Uppsala, Sweden).

The tissue filler may comprise a relatively insoluble cross-linked hyaluronic acid component within a soluble fluid component. The cross-linked hyaluronic acid component may comprise cohesive particles. The soluble fluid component may comprise free hyaluronic acid (non cross-linked) or any other fluid component which can facilitate the delivery of the tissue filler composition through fine bore needles. The concentration of hyaluronic acid (including both cross-linked and non cross-linked hyaluronic acid components) can vary from about 4.5 to about 40 mg/mL, about 4.5 to about 35 mg/mL, about 4.5 to about 30 mg/mL, about 4.5 to about 25 mg/mL, about 4.5 to about 20 mg/mL, about 4.5 to about 15 mg/mL, about 4.5 to about 10 mg/mL. The concentration of hyaluronic acid (including both cross-linked and non cross-linked hyaluronic acid components) can be about 10 mg/mL, about 12 mg/mL, about 14 mg/mL, about 16 mg/mL, about 18 mg/mL, about 20 mg/mL, about 22 mg/mL, or about 24 mg/mL. The non cross-linked hyaluronic acid component may comprise from about 30% to about 100% of the total hyaluronic acid, or about 40-100%, about 40-99%, about 40-98%, about 40-95%, about 40-90%, about 40-85%, about 40-80%, about 40-75%, about 40-70%, about 40-65%, about 40-60%, about 40-55%, about 40-50%, about 40-45%, about 50-100%, about 50-99%, about 50-98%, about 50-95%, about 50-90%, about 50-85%, about 50-80%, about 50-75%, about 50-70%, about 50-65%, about 50-60%, about 50-55%, about 60-100%, about 60-99%, about 60-98%, about 60-95%, about 60-90%, about 60-85%, about 60-80%, about 60-75%, about 60-70% of the total hyaluronic acid content in the composition. The cross-linked hyaluronic acid component may comprise from about 1% to about 20% of the total hyaluronic acid content in the composition. In certain embodiments, the cross-linked hyaluronic acid component comprises from about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%. In certain embodiments, the cross-linked hyaluronic acid component comprises from about 1% to about 12%, about 3% to about 10%, or about 4% to about 10%.

In certain embodiments, the tissue filler comprises cross-linked hyaluronic acid particles within a fluid component, the particles may be the same or different sizes. The particles may be sized to enable them to pass through a fine needle bore such as a 27-40G needle. The particle sizes may range from 100-1000 microns, or about 200-1000 microns, about 250-1000 microns, about 300-1000 microns, about 350-1000 microns, about 400-1000 microns about 450 -1000 microns, about 500-1000 microns, about 550-1000 microns, about 600-1000 microns, about 650-1000 microns, about 700-1000 microns, about 200-900 microns, about 250-900 microns, about 300-900 microns, about 350-900 microns, about 400-900 microns about 450-900 microns, about 500-900 microns, about 550-900 microns, about 600-900 microns, about 650-900 microns, about 700-900 microns, 200-800 microns, about 250-800 microns, about 300-800 microns, about 350-800 microns, about 400-800 microns about 450 -800 microns, about 500-800 microns, about 550-800 microns, about 600-800 microns, about 650-800 microns, or about 700-800 microns.

For example, Emervel® comprises about 20 mg/ml hyaluronic acid having a particle size of about 400 microns and is suitable for injection into the mid-to-deep dermis. Restylane® comprises about 20 mg/ml hyaluronic acid having a particle size of about 400 microns and is suitable for injection into mid-to-deep dermis. ^{Perlane}® comprises about 20 mg/ml hyaluronic acid having a particle size of between about 750 microns and about 1000 microns and is suitable for injection into deep dermis and/or superficial subcutis. Radiesse® comprises about 30% calcium hydroxylapatite microspheres/70% carrier gel and is suitable for subdermal injections.

In some embodiments, the tissue filler is substantially stable at room temperature for, e.g., about 3 months, about 6 months, about 9 months, about 12 months, about 15 months, about 18 months, about 21 months, about 24 months, about 27 months, about 30 months, about 33 months, or about 36 months. In other aspects of this embodiment, a dermal filler composition is substantially stable at room temperature for, e.g., at least 3 months, at least 6 months, at least 9 months, at least 12 months, at least 15 months, at least 18 months, at least 21 months, at least 24 months, at least 27 months, at least 30 months, at least 33 months, or at least 36 months. In other aspects of this embodiment, a tissue filler composition is substantially stable at room temperature for, e.g., about 3 months to about 12 months, about 3 months to about 18 months, about 3 months to about 24 months, about 3 months to about 30 months, about 3 months to about 36 months, about 6 months to about 12 months, about 6 months to about 18 months, about 6 months to about 24 months, about 6 months to about 30 months, about 6 months to about 36 months, about 9 months to about 12 months, about 9 months to about 18 months, about 9 months to about 24 months, about 9 months to about 30 months, about 9 months to about 36 months, about 12 months to about 18 months, about 12 months to about 24 months, about 12 months to about 30 months, about 12 months to about 36 months, about 18 months to about 24 months, about 18 months to about 30 months, or about 18 months to about 36 months.

In some embodiments, the tissue filler medium is degradable and degrades in vivo in about 1 month, 2 months, 3 months, about 3 months to about 12 months, about 3 months to about 18 months, about 3 months to about 24 months, about 6 months to about 12 months, about 6 months to about 18 months, about 9 months to about 12 months, about 9 months to about 18 months, about 12 months to about 18 months.

Additional agents may be combined with the tissue filler. The agent combined with the polymer may comprise an anaesthetizing agent or a pain-killing agent (e.g., lidocaine), or a vitamin (e.g., vitamin C). The additional agent may comprise a biologically active component such as growth factors, peptides and cells.

### ii) Light-Absorbing Molecules (LAMs)

In some embodiments, the PBM compositions of the present disclosure comprise one or more light-absorbing molecules, which can be considered exogenous, e.g., are not naturally present in skin or tissue. Suitable light-absorbing molecules can be light-absorbing molecules, chromophores, fluorophores or fluorochromes such as fluorescent dyes (or stains), although other dye groups or dyes (biological and histological dyes, food colorings, carotenoids, naturally occurring fluorescent and other dyes) can also be used. Suitable light-absorbing molecules can be those that are Generally Regarded As Safe (GRAS). Light-absorbing molecules which are not well tolerated by the skin or other tissues can be included in the PBM compositions in an encapsulated, or chemically modified form.

In certain embodiments, the PBM compositions of the present disclosure comprise a light-absorbing molecule that undergoes partial or complete photobleaching upon application of light. By photobleaching is meant a photochemical destruction of the light-absorbing molecule which can generally be visualized as a loss of color. In some embodiments, the light-absorbing molecule absorbs at a wavelength in the range of the visible spectrum, such as at a wavelength of about 380 nm and about 800 nm, about 380 nm and about 700 nm, about 400 nm and about 700 nm, or about 380 nm and about 600 nm. In these embodiments, the first light-absorbing molecule is not activated by UV light. In other embodiments, the light-absorbing molecule absorbs at a wavelength of about 200 nm and about 800 nm, about 200 nm and about 700 nm, about 200 nm and about 600 nm, or about 200 nm and about 500 nm. In one embodiment, the light-absorbing molecule absorbs at a wavelength of about 200 nm and about 600 nm. In some embodiments, the light-absorbing molecule absorbs light at a wavelength of about 200 nm and about 300 nm, about 250 nm and about 350 nm, about 300 nm and about 400 nm, about 350 nm and about 450 nm, about 400 nm and about 500 nm, about 400 nm and about 600 nm, about 450 nm and about 650 nm, about 600 nm and about 700 nm, about 650 nm and about 750 nm or about 700 nm and about 800 nm.

The PBM compositions disclosed herein may include at least one additional light-absorbing molecule. Combining light-absorbing molecules may increase photo-absorption by the combined dye molecules and enhance absorption and photo-biomodulation selectivity. This creates multiple possibilities of generating new photosensitive, and/or selective light-absorbing molecule mixtures.

When such multi-light-absorbing molecules compositions are illuminated with light, energy transfer can occur between the light-absorbing molecules. This process, known as resonance energy transfer, is a photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule). The efficiency and directedness of resonance energy transfer depends on the spectral features of donor and acceptor light-absorbing molecules.

In certain embodiments, the PBM compositions of the present disclosure further comprises a second light-absorbing molecule. In some embodiments, the first light-absorbing molecule has an emission spectrum that overlaps at least about 80%, at least about 50%, at least about 40%, at least about 30%, at least about 20%, or at least about 10% with an absorption spectrum of the second light-absorbing molecule. In one embodiment, the first light-absorbing molecule has an emission spectrum that overlaps at least about 20% with an absorption spectrum of the second light-absorbing molecule. In some embodiments, the first light-absorbing molecule has an emission spectrum that overlaps at least about 1-10%, at least about 5-15%, at least about 10-20%, at least about 15-25%, at least about 20-30%, at least about 25-35%, at least about 30-40%, at least about 35-45%, at least about 50-60%, at least about 55-65% or at least about 60-70% with an absorption spectrum of the second light-absorbing molecule.

As discussed above, the application of light to the PBM compositions of the present disclosure can result in a cascade of energy transfer between the tissue filler and the light-absorbing molecule or between the multi-light-absorbing molecules. In certain embodiments, such a cascade of energy transfer provides emission of photons from inside the soft tissue that can travel deeper than if the photons were travelling transdermally.

Optionally, when the PBM composition comprises a first and a second light-absorbing molecules, the first light-absorbing molecule is present in an amount of about 0.001-40% per weight of the PBM composition, and the second light-absorbing molecule is present in an amount of about 0.001-40% per weight of the PBM composition.

Particularly useful combinations of xanthene dyes include but are not limited to: Fluorescein+Eosin Y; Fluorescein+Eosin Y+Rose Bengal; Fluorescein+Eosin Y+Phloxine B; Eosin Y+Rose Bengal; Eosin Y+Phloxine B; Eosin Y+Erythrosine; Fluorescein+Erythrosine B+Eosin Y; Eosin Y+Erythrosine B+Rose Bengal; Eosin Y+Erythrosine B+Phloxine B; Fluorescein+Eosin Y+Erythrosine B+Rose Bengal; and Fluorescein+Eosin Y+Erythrosine B+Phloxine B.

It is thought that at least some of these combinations have a synergistic effect at certain concentration ratios within the composition. For example, at certain concentration ratios and with an appropriate activating light, Eosin Y can transfer energy to Rose Bengal, Erythrosin B or Phloxine B when activated.

The synergistic effect may be apparent by the composition having a light absorption spectrum which spans a broader range of wavelengths compared to an individual light absorption spectrum of one of the individual light-absorbing molecules in the composition, when the individual light-absorbing molecules and the composition are activated by the same activating light (light having substantially the same emission spectra). This may confer on the composition the ability to be activated by a broader range of activating light wavelengths, for example by white light avoiding the need for a precise wavelength of activating light.

The synergistic effect may also be evident through the composition having a light emission spectrum which spans a broader range of wavelengths compared to an individual light absorption spectrum of one of the individual light-absorbing molecules in the composition, when the individual light-absorbing molecules and the composition are activated by the same activating light. This absorbed and re-emitted light spectrum is thought to be transmitted throughout the composition, and also to be transmitted into the site of treatment. This emitted spectrum will then illuminate the target tissue with different penetration depth, which may confer on the target tissue beneficial therapeutic effects. By emitting a broader range of wavelengths, a broader range of therapeutic effects can be achieved. The emitted wavelength can be fine-tuned using different light-absorbing molecules combinations and concentrations.

The synergistic effect may also be evident through the composition having a higher light absorption or emission peak compared to an individual light absorption/emission peak of one of the individual light-absorbing molecules in the composition, when the individual light-absorbing molecules and the composition are activated by the same activating light. The ability to absorb and emit higher levels of photons may have a therapeutic effect in certain applications. Furthermore, less concentration of an individual light-absorbing molecules may be required to achieve a certain power density. Higher power densities can equate to shorter treatment times.

By means of synergistic effects of the xanthene dye combinations in the composition, xanthene dyes which cannot normally be activated by an activating light (such as a blue light) can be activated through energy transfer from xanthene dyes which are activated by the activating light. In this way, the different properties of photoactivated xanthene dyes can be harnessed and tailored according to the cosmetic or the medical therapy required.

One or more of the light-absorbing molecules may photobleach during illumination. This can be a visible confirmation of ‘dose’ delivery. As the chromophores photobleach, they emit less fluorescence over time. At the same time, they also absorb less of the activating light over time and so the tissues receive increasingly higher amounts of the activating light. In this way, the light-absorbing molecules modulate exposure of the tissue to the light which may provide a somewhat protective effect.

In certain embodiments, the PBM compositions of the present disclosure comprise at least two light-absorbing molecule (e.g., Eosin Y and Fluorescein). In some embodiments, the PBM compositions comprise at least 3, at least 4, at least 5, at least 6, at least 7, at least 8, at least 9, or at least 10 light different light-absorbing molecules.

In some embodiments, the light-absorbing molecule is selected such that their emitted fluorescent light, on photoactivation, is within one or more of the blue, green, yellow, orange, red and infrared portions of the electromagnetic spectrum, for example having a peak wavelength within the range of about 450 nm to about 500 nm, 490 nm to about 800 nm, or about 470 nm to about 700 nm. In certain embodiments, the emitted fluorescent light has a peak power density of between 0.005 mW/cm² to about 10 mW/cm², or about 0.5 mW/cm² to about 5 mW/cm².

Suitable light-absorbing molecules that may be used in the PBM compositions of the present disclosure include, but are not limited to the following:
iii) Chlorophyll dyes—Exemplary chlorophyll dyes include but are not limited to chlorophyll a; chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.
iv) Xanthene derivatives—Exemplary xanthene dyes include but are not limited to Eosin B (4′,5′-dibromo,2′,7′-dinitr-o-fluorescein, dianion); eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.
v) Methylene blue dyes—Exemplary methylene blue derivatives include but are not limited to 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-a-mino-phenothiazine; and 1,9-dimethyl-3-diethylamino-7-dibutyl-amino-phenot-hiazine.
vi) Azo dyes—Exemplary azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.
vii) In some aspects of the disclosure, the one or more light-absorbing molecules of the PBM composition disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Saffranin O), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments, the PBM compositions of the present disclosure includes Eosin Y as a first light-absorbing molecule and any one or more of Rose Bengal, Fluorescein, Erythrosine, Phloxine B, chlorophyllin as a second light-absorbing molecule. It is believed that these combinations have a synergistic effect as they can transfer energy to one another when activated due in part to overlaps or close proximity of their absorption and emission spectra. This transferred energy is then emitted as fluorescence or leads to production of reactive oxygen species. This absorbed and re-emitted light is thought to be transmitted throughout the PBM composition, and also to be transmitted into the site of treatment.

In further embodiments, the PBM composition includes the following combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. Other synergistic light-absorbing molecule combinations are also possible.

In certain embodiments, the light-absorbing molecule is activated by light having a wavelength in the visible range. In certain implementations, the light-absorbing molecule does not absorb light in the UV range of the electromagnetic spectrum. In certain embodiments, when activated, the light-absorbing molecule can emit light having a wavelength in the visible range. The emitted light can be within one or more of the violet, blue, green, yellow, orange or red portions of the electromagnetic spectrum. Alternatively, the light-absorbing molecule can emit in the infrared range. In certain embodiments, the light-absorbing molecule can be photobleached after illumination of the area. In certain embodiments, the light-absorbing molecule may be photobleached after 5, 10, 15, 20, 25, 30, 35, 40, 45, 50, 55, 60 or 65 minutes of illumination. The illumination may be continuous or pulsed. The illumination may comprise re-illumination of the PBM composition or the area after a few hours or days. In certain embodiments, by means of the photobleaching, photosensitivity is reduced or avoided. In certain embodiments, the light-absorbing molecule is not a photosensitizing agent requiring to be metabolized. In certain embodiments, the area is illuminated at the same time as or immediately after intradermal or subdermal administration. In other words, there is no incubation period between administering of the PBM composition and illumination.

In certain embodiments, the illumination is for a time sufficient to activate the light-absorbing molecule. In certain embodiments, the illumination is for a time sufficient to photobleach the light-absorbing molecule. In certain embodiments, the illumination is for 1-30 seconds, 15-45 seconds, 30-60 seconds, 0.75-1.5 minutes, 1-2 minutes, 1.5-2.5 minutes, 2-3 minutes, 2.5-3.5 minutes, 3-4 minutes, 3.5-4.5 minutes, 4-5 minutes, 5-10 minutes, 10-15 minutes, 15-20 minutes, 20-25 minutes, or 25-30 minutes. The treatment time may range up to about 90 minutes, about 80 minutes, about 70 minutes, about 60 minutes, about 50 minutes, about 40 minutes or about 30 minutes.

In certain embodiments, the light-absorbing molecule is compatible with the tissue filler in that the light accepting molecule does not chemically and physically affect the properties of the tissue filler and, reversibly, the tissue filler does not affect the chemical and the physical properties of the light-absorbing molecule.

In some embodiments, the light-absorbing molecule maintains and/or ameliorates the viscoelastic properties, cohesive properties, elastic properties, stability properties, safety properties, and/or plasticity properties of the tissue filler.

iii) Methods of Use

In some embodiments, the present disclosure provides PBM compositions which can be injected or implanted within soft tissue as tissue fillers. Such PBM compositions comprise at least one light-absorbing molecule and at least one tissue filler which can be activated by light having an appropriate wavelength causing the light-absorbing molecule to emit light. In some instances, the activated tissue filler may also emit light. The activating and/or emitted light may have a therapeutic effect on its own. The activated light-absorbing molecule, activated tissue filler and/or the light may also lead to the photochemical activation of other agents contained in the composition or at a treatment site.

Without being bound to theory, it is thought that fluorescent light emitted by photoactivated tissue filler or photoactivated light-absorbing molecules may have therapeutic properties due to its femto-, pico- or nano-second emission properties which may be recognized by biological cells and tissues, leading to favorable biomodulation. Furthermore, the emitted fluorescent light has a longer wavelength and hence a deeper penetration into the tissue than the activating light. Irradiating intradermal, subdermal or other soft tissue with such a broad range of wavelengths, including in some embodiments the activating light which passes through the PBM composition, may have different and complementary effects on the cells and tissues.

The PBM compositions of the present disclosure have numerous uses. Without being bound by theory, the PBM compositions of the present disclosure may be used for skin rejuvenation. The PBM compositions of the present disclosure may promote wound healing or tissue repair. The PBM compositions of the present disclosure may provide cosmetic enhancement of soft tissue. The PBM compositions of the present disclosure may inhibit or treat scarring. The PBM compositions of the present disclosure may stimulate collagen synthesis. This collagen synthesis may be useful for management of wounds, tissue repair, skin rejuvenation, or cosmetic enhancement of soft tissue.

It is an objective of the present technology to provide a method for providing biophotonic therapy to a wound, where the method promotes collagen synthesis in the wound. It is an objective of the present disclosure to provide a method for providing biophotonic therapy to a wound, where the method prevents scar formation. It is also an objective of the present disclosure to provide a method for providing biophotonic therapy to skin tissue, wherein the method is used for promoting skin rejuvenation. It is also an objective of the present disclosure to provide a method for providing biophotonic therapy to skin tissue, wherein the method is used for promoting collagen synthesis.

In some embodiments, the PBM compositions of the present disclosure may be used to minimize the appearances of fine lines in skin that would not otherwise be minimized by conventional tissue filling methods that are performed without the photo-stimulation (i.e., without illumination by an external light source). In some implementations of these embodiments, the minimization of the appearance of the fine lines is long-lasting, delaying the re-appearance of the fine lines for months or for years.

In the methods disclosed herein, the PBM composition may be prepared by mixing a tissue filler composition and a light-absorbing composition prior to injection into the tissue. In some embodiments, the light-absorbing molecule composition is added to the tissue filler composition during the tissue filler manufacturing process (e.g., during the step of cross-linking the tissue filler). In some implementations, the light-absorbing molecules are dispersed between the chains of the tissue filler. For example, the light-absorbing molecules are dispersed in the space created between the cross-linked chains of hyaluronic acid.

In the methods disclosed herein, the PBM composition can be administered by injection or implantation (e.g., using a syringe and needle, etc.) into or underneath soft tissue at a treatment site (e.g., subcutaneous administration, intradermal administration, subdermal administration). A skilled person can select an appropriate needle bore size according to the soft tissue to be treated. In intradermal applications, needle gauges of 27G to 40 G can be used, typically 30 or 32G. The higher the gauge size, the finer the needle bore. The PBM compositions of the disclosure can also be injected or implanted superficially, such as, for example, within the papillary layer of the dermis, or can be injected or implanted within the reticular layer of the dermis.

The PBM composition can be administered subcutaneously, intradermally or subdermally into the dermis in a continuous fashion (e.g., linear threading) or using pin pricks to form discrete pockets of the PBM composition subcutaneously, intradermally or subdermally (e.g. serial puncture technique). The PBM composition can be illuminated at the same time as administering the composition to the soft tissue site, or after administration.

In certain embodiments, about 1 ml of the PBM composition is administered subcutaneously, intradermally or subdermally. In certain embodiments, less than about 1 ml of the PBM composition is administered intradermally or subdermally. In certain embodiments, between about 0.1 ml and about 0.2 ml, between about 0.2 ml and about 0.3 ml, between about 0.3 ml and about 0.4 ml, between about 0.4 ml and about 0.5 ml, between about 0.5 ml and about 0.6 ml, between about 0.6 ml and about 0.7 ml, between about 0.7 ml and about 0.8 ml, between about 0.8 ml and about 0.9 ml, between about 0.9 ml and about 1.0 ml of the PBM composition is administered subcutaneously, intradermally or subdermally.

In the methods of the present disclosure, the PBM composition may be illuminated transdermally (from outside the body) or from within the soft tissue (e.g. using an optical fiber or a light duct). In certain embodiments, the PBM composition may itself form a light duct from the skin surface to the PBM composition. To form such a light duct, a trail of the PBM composition is formed from the skin puncture point to the PBM composition within the soft tissue. In the methods of the present disclosure, the PBM composition may be illuminated at the same time as or immediately following administration of the PBM composition to the intracorporeal area.

In the methods of the present disclosure, any source of actinic light can be used. Any type of halogen, LED or plasma arc lamp or laser may be suitable. The primary characteristic of suitable sources of actinic light will be that they emit light in a wavelength (or wavelengths) appropriate for activating the one or more light-absorbing molecules present in the PBM composition. In one embodiment, an argon laser is used. In another embodiment, a potassium-titanyl phosphate (KTP) laser (e.g., a GreenLightTM laser) is used. In another embodiment, sunlight may be used. In yet another embodiment, a LED photocuring device is the source of the actinic light. In yet another embodiment, the source of the actinic light is a source of light having a wavelength between about 200 nm and about 800 nm. In another embodiment, the source of the actinic light is a source of visible light having a wavelength between about 400 nm and about 600 nm or between about 400 nm and about 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 1 mW/cm² to about 200 mW/cm². Suitable power densities for laser light sources are in the range from about 0.5 mW/cm² to about 0.8 mW/cm².

In some embodiments of the methods of the present disclosure, the light has an energy at the subject's skin, wound or mucosa surface of between about 1 mW/cm² and about 500 mW/cm², between about 1 mW/cm² and about 300 mW/cm², or between about 1 and about 200 mW/cm², wherein the energy applied depends at least on the condition being treated, the wavelength of the light, the distance of the subject's skin from the light source, and the thickness of the PBM composition. In certain embodiments, the light at the subject's skin is between about 1 mW/cm² and about 40 mW/cm², or between about 20 mW/cm² and about 60 mW/cm², or between about 40 mW/cm² and about 80 mW/cm², or between about 60 mW/cm² and about 100 mW/cm², or between about 80 mW/cm² and about 120 mW/cm², or between about 100 mW/cm² and about 140 mW/cm², or between about 120 mW/cm² and about 160 mW/cm², or between about 140 mW/cm² and about 180 mW/cm², or between about 60 mW/cm² and about 200 mW/cm², or between about 110 mW/cm² and about 240 mW/cm², or between about 110 mW/cm² and about 150 mW/cm², or between about 190 mW/cm² and about 240 mW/cm².

In certain embodiments, the light-absorbing molecules in the PBM composition can be photoexcited by ambient light including from the sun and overhead lighting. In certain embodiments, the light-absorbing molecules can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs etc. and indirect sunlight.

The duration of the exposure to actinic light required will be dependent on depth beneath the skin surface of the PBM composition; the thickness, density and components of the intervening tissue; the type of intervening tissue, the concentration of light-absorbing molecule within the PBM composition, the power density, wavelength and bandwidth of the light source, the thickness of the PBM composition, and the treatment distance from the light source. The illumination of the treated area by fluorescence may take place within seconds or even fragments of seconds, but a prolonged exposure period is beneficial to exploit the synergistic effects of the absorbed, reflected and reemitted light on the composition of the present disclosure and its interaction with the tissue being treated.

In one embodiment, the time of exposure to actinic light of the tissue in which the PBM composition has been administered is a period between 1 minute and 5 minutes. In another embodiment, the time of exposure to actinic light of the tissue in which the PBM composition has been administered is a period between 1 minute and 5 minutes. In some other embodiments, the PBM composition is illuminated for a period between 1 minute and 3 minutes. In certain embodiments, light is applied for a period of between about 1 second and about 30 seconds, between about 15 seconds and about 45 seconds, between about 30 seconds and about 60 seconds, between about 0.75 minute and about 1.5 minutes, between about 1 minute and about 2 minutes, between about 1.5 minute and about 2.5 minutes, between about 2 minutes and about 3 minutes, between about 2.5 minutes and about 3.5 minutes, between about 3 minutes and about 4 minutes, between about 3.5 minutes and about 4.5 minutes, between about 4 minutes and about 5 minutes, between about 5 minutes and about 10 minutes, between about 10 minutes and about 15 minutes, between about 15 minutes and about 20 minutes, between about 20 minutes and about 25 minutes, or between about 20 minutes and about 30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², about 15 J/cm² to 25 J/cm², or about 10 J/cm² to about 20 J/cm².

The method may be repeated as necessary. For example, in certain embodiments where the tissue filler is a biodegradable material, the method may be repeated close to or after full degradation of the tissue filler.

The PBM composition may be administered at regular intervals such as once a week, or at an interval deemed appropriate by the physician. Alternatively, once administered, the PBM composition may be light activated at regular intervals until exhaustion of the light-absorbing molecule or until the depletion of the tissue filler.

In some instances, the frequency of administration of the PBM compositions as defined herein may depend on the type and/or the quantity of the tissue filler that is initially administered. Tissue fillers can be re-administered as early as a few months and as late as 2 years depending on their thickness, viscosity and elasticity. As such, the question of administrations per unit time may depend on the type of tissue filler, the placement, the depth of tissue-site, and the quantity administered.

In some embodiments, the present technology also provides for method to achieve biomodulation, the method comprising injecting a PBM composition of the present technology into a soft tissue as well as applying a PBM composition onto the injected soft tissue and illuminating the injected area with actinic light for a time sufficient to photoactivate the light-absorbing molecules of the injected and/or of the applied PBM composition.The present disclosure also provides kits for preparing and/or administering any of the PBM compositions of the present disclosure. The kit may include a container comprising the PBM composition of the present disclosure. The container may be light impermeable, air-tight and/or leak resistant. Exemplary containers include, but are not limited to, syringes, vials, or pouches. In some embodiments, the tissue filler and the light-absorbing molecule are provided in a same container (e.g., syringe), the content of which is ready for administration. In some other embodiments, the tissue filler and the light-absorbing molecule are provided in separate containers (e.g., syringes) that are to be mixed by the user prior to administration. In some embodiments, the kit comprises a tissue filler provided in a single container (e.g., syringe), the content of which is ready for administration. In some embodiments, the kit comprises a mixing device and/or a handheld injection device.

In other embodiments, the kit comprises a systemic or topical drug for augmenting the treatment of the PBM composition. For example, the kit may include a systemic or topical antibiotic or hormone treatment for acne treatment or wound healing.

Written instructions on how to use the PBM composition in accordance with the present disclosure may be included in the kit, or may be included on or associated with the containers comprising the PBM compositions of the present disclosure.

In certain embodiments of the kit, the kit may further comprise a light source such as a portable light with a wavelength appropriate to activate the tissue filler and/or the light-absorbing molecule in the PBM composition. The portable light may be battery operated or re-chargeable.

In certain embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are provided to illustrate the practice of various embodiments of the present technology. They are not intended to limit or define the entire scope of this technology. It should be appreciated that the technology is not limited to the particular embodiments described and illustrated herein but includes all modifications and variations falling within the scope of the disclosure as defined in the appended embodiments.

### Example 1

### Experimental Setup for Assessment of Photo-Biomodulation on Collagen Production in Animal Skin

The effects of photo-biomodulation compositions of the present technology on collagen production in the dermis and on incisional wound healing were assessed in a porcine model. The pig models were injected with photo-biomodulation (PBM) compositions according to various embodiments of the present disclosure.

The PBM compositions were prepared as follows: a 1 cc syringe was filled with a tissue filler composition and another 1 cc syringe was filled with the light-absorbing molecule composition. The content of the two syringes were mixed together via a connector right before injection into the skin of the pigs. The light-absorbing molecules and tissue fillers used in the preparation of the PBM compositions were as follows:
viii) 0.012% of Eosin Y (Light-absorbing molecule #1 (LAM 1))
ii) 0.012% of Eosin Y and Fluorescein (Light-absorbing molecule #2 (LAM 2))
Tissue filler selected from:
1. Emervel® Classic (Galderma, Lausanne, Switzerland),
2. Emervel® Volume (Galderma, Lausanne, Switzerland),
3. Perlane® (Galderma, Lausanne, Switzerland),
4. Radiesse® (Merz Aesthetics, NC, USA),
5. Restylane® (Galderma, Lausanne, Switzerland),
6. Restylane® Fine Line (Galderma, Lausanne, Switzerland).

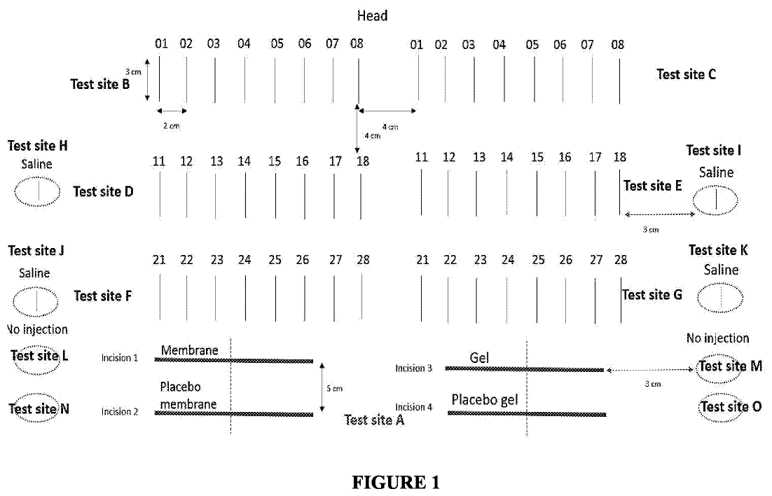

Each animal was placed in ventral recumbency. The hair was removed from the treatment area on the back of the animal. The surgical site was prepared with topical cleaning using a neutral (non-antibacterial nor antiseptic) soap, rinsed with sterile saline followed by an application of 70% isopropyl alcohol. Ten areas were drawn with a skin marker or tattooed to delineate the sites of injection and incision. The pigs were injected with the PBM compositions as outlined in Table 1 and according to the injection program illustrated in FIG. 1.

TABLE 1
PBM compositions injected and light used for phototreatment

| | PBM Compositions | | |
|---|---|---|---|
| Animal | Dermal Filler | Light-absorbing molecule | Light |
| 1 | Emervel ® Classic | Eosin Y | Blue |
| | | Eosin Y | Green |
| | | Eosin Y + Fluorescein | Blue |
| | | Eosin Y + Fluorescein | Green |
| 2 | Entervel ® Volume | Eosin Y | Blue |
| | | Eosin Y | Green |
| | | Eosin Y + Fluorescein | Blue |
| | | Eosin Y + Fluorescein | Green |
| 3 | Perlane ® | Eosin Y | Blue |
| | | Eosin Y | Green |
| | | Eosin Y + Fluorescein | Blue |
| | | Eosin Y + Fluorescein | Green |
| 4 | Radiesse ® | Eosin Y | Blue |
| | | Eosin Y | Green |
| | | Eosin Y + Fluorescein | Blue |
| | | Eosin Y + Fluorescein | Green |
| 5 | Restylane ® | Eosin Y | Blue |
| | | Eosin Y | Green |
| | | Eosin Y + Fluorescein | Blue |
| | | Eosin Y + Fluorescein | Green |
| 6 | Restylane ® Fine | Eosin Y | Blue |
| | Line | Eosin Y | Green |
| | | Eosin Y + Fluorescein | Blue |
| | | Eosin Y + Fluorescein | Green |

Klox Thera® Lamp was used for the illumination of the injected skin section and activation of the PBM composition injected therein. Specifically, Klox Thera® lamp with Blue LEDs (B LED) and Klox Thera® lamp with Green LEDs (G LED) were used on the injected samples. The wavelengths of the green or blue light emitted ranged between 420 nm to 490 nm or with a wavelength around 566 nm. The irradiance or power density of the light was between 100 mW/cm² and 150 mW/cm² at a distance of 5 cm from the light source with a radiant fluences (or dose) during a single treatment for 5 minutes of 33 J/cm² to 45 J/cm².

Forty-six subcutaneous injections (100 μl to 300 μl each) were performed. Positions of the injections are as presented in FIG. 1 (areas B to K). Four areas were left without any injection (FIG. 1, areas L to O). Four skin incisions of ˜8 cm length were performed on each animal. Each incision were rinsed with sterile saline and dried with sterile gauze. The incisions were then be sutured using 4-0 Ethicon. Positions of the incisions are presented in FIG. 1 (area A). Positions may be adjusted based on the anatomy and size of each animal.

One histological section (approximately 5 micron thick) was taken through the center of each illuminated injection site, including margins of normal skin. Sections were then embedded in paraffin. Paraffin blocks were analyzed for the following markers of collagen synthesis: Decorin, Collagen III, Ki67. A histological assessment of the paraffin blocks was also performed.

The duration of the study was chosen based on the normal wound healing process estimated to be between 20 and 25 days as well as collagen synthesis which can take several weeks in the dermis to occur. The pig modes used for the study were as defined in Table 2. Table 3 presents the grading system for the histological assessment:

TABLE 2
characteristics of animal model

| Species: | Sus scrofa |
|---|---|
| Strain: | Hybrid farm pigs (Landrace-Yorkshire) |
| Condition: | Non diseased |
| Source: | Triporc Inc. |
| Age at implant: | Young adult |
| Weight at implant: | 20 ± 5 kg |
| Sex: | Female |
| Number: | 12 + 1 spares |
| Strain: | Hybrid farm pigs (Landrace-Yorkshire) |

TABLE 3
Grading for histological assessment
Histologic Scoring System

| Feature Scored | Score | Description |
|---|---|---|
| Epidermis | 0 | None (normal aspect) |
| alterations | 1 | Mild (mild degeneration of keratinocytes -mild swelling of the cells) |
| | 2 | Moderate (focal area of disepitelization cellular necrosis, inflammatory |
| | | infiltration); ⅓ of adnexa damaged |
| | 3 | Severe (large area to total disepitelization, oedema of dermal papillae, |
| | | severe inflamm. inf.); ⅔ of adnexa damaged |
| Dermis alterations | 0 | None (normal aspect) |
| | 1 | Mild dermal oedema, collagen disorganization and lymphatic vessels dilation |
| | 2 | Moderate dermal oedema, collagen areas of disorganization with loss of criss- |
| | | crossing aspect of collagen fibers, mod lymphatic ectasia |
| | 3 | Severe dermal oedema, with or without inflammation, with some collagen areas |
| | | with signs of hyalinosis, shrinked aspect of collagen fibres, areas of fusion |
| | | of the fibers, and amorphous coartation, severe lymphatic ectasia. |
| Lymphatic vessels | 0 | Non evaluable |
| status | 1 | Mild lymphatic vessels dilation, without lymph. |
| | 2 | Mod lymphatic ectasia, with some traces of lymphatic fluid. |
| | 3 | Severe lymphatic ectasia with lymphostasis. |
| Collagen fibres | 0 | 100% of criss-crossing fibres. |
| organization | 1 | 76-100% of criss-crossing fibres - absence of areas of coartation/hyalinosis |
| | 2 | 51-75% of criss-crossing fibres - some areas of mild coartation/hyalinosis |
| | | and general oedema |
| | 3 | 26-50% only of criss-crossing fibres - various areas of coartation/hyalinosis |
| | | and general oedema |
| Masson Trichrome | 0 | 1.15-1.50 mm dermal thickening; 100% of criss-crossing fibres. |
| | 1 | 1.50-1.60 mm dermal thickening; 76-100% of criss-crossing fibres - absence of |
| | | areas of coartation/hyalinosis |
| | 2 | 1.60-1.70 mm dermal thickening; 51-75% of criss-crossing fibres - some areas |
| | | of mild coartation/hyalinosis and general oedema |
| | 3 | 1.70-2.00 mm dermal thickening; 26-50% only of criss-crossing fibres - various |
| | | areas of coartation/hyalinosis and general oedema |

### Example 2

### Assessment of Injected Photo-Biomodulation Compositions on Collagen Production and Management of Wounds

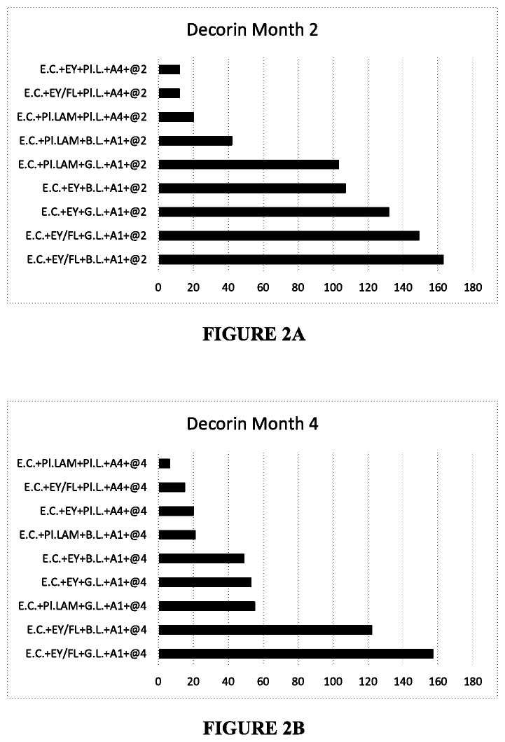

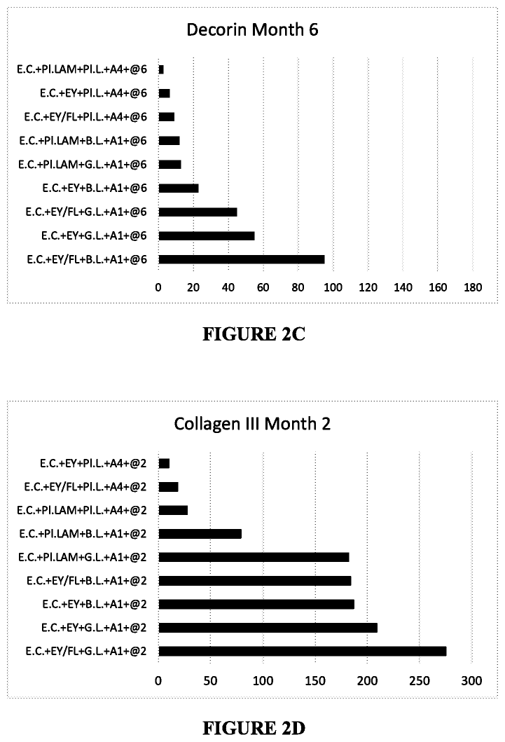

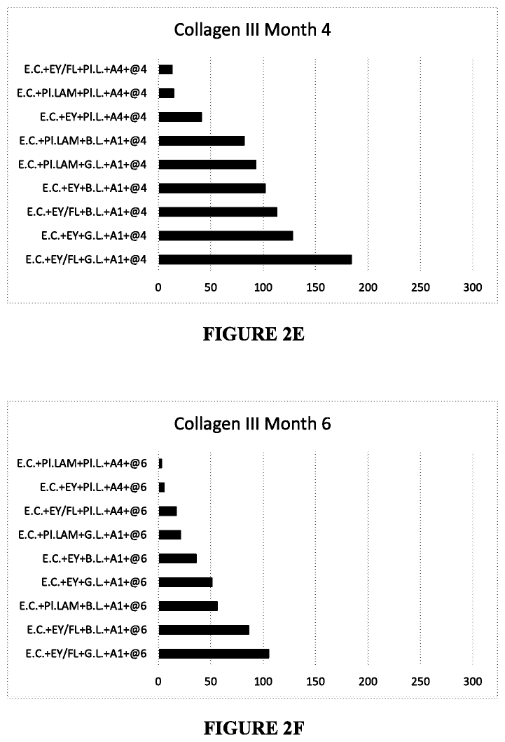

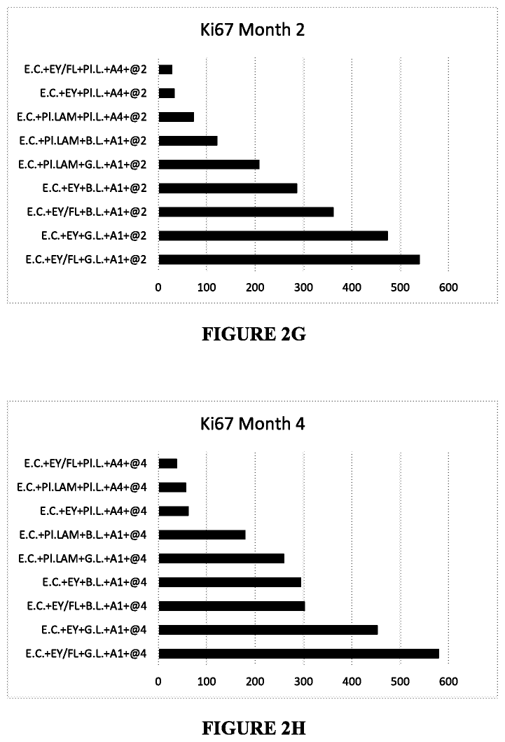

The skin samples that were injected and phototreated as outlined in Example 1 were assessed for expression of the following collagen markers: Decorin, Collagen III and Ki67 at 2 months post-phototreatment, at 4 months post-phototreatment and at 6 months post-phototreatment. Results are presented in FIGS. 2 to 19. Table 4 outlines the correspondence between the data presented in each of FIGS. 2 to 7 to the injected animals. Each tissue filler was tested in the pig model.

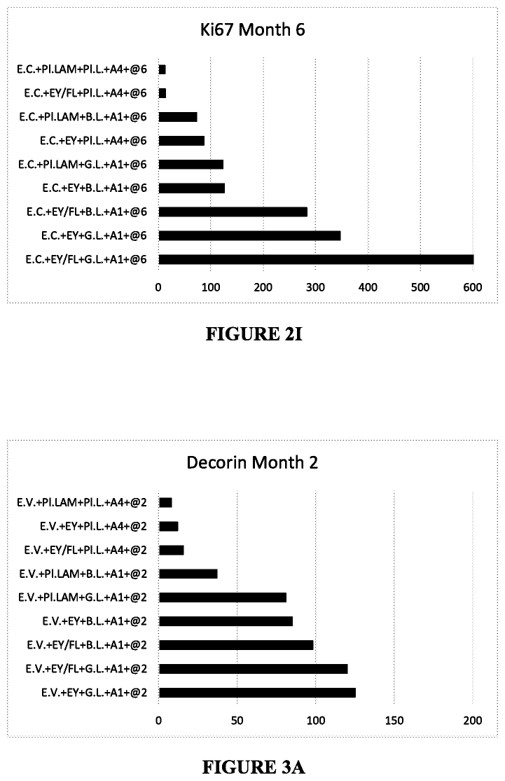

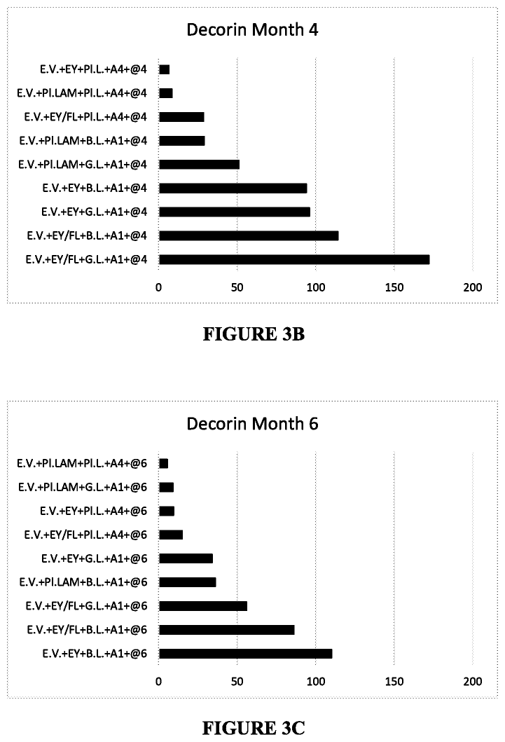

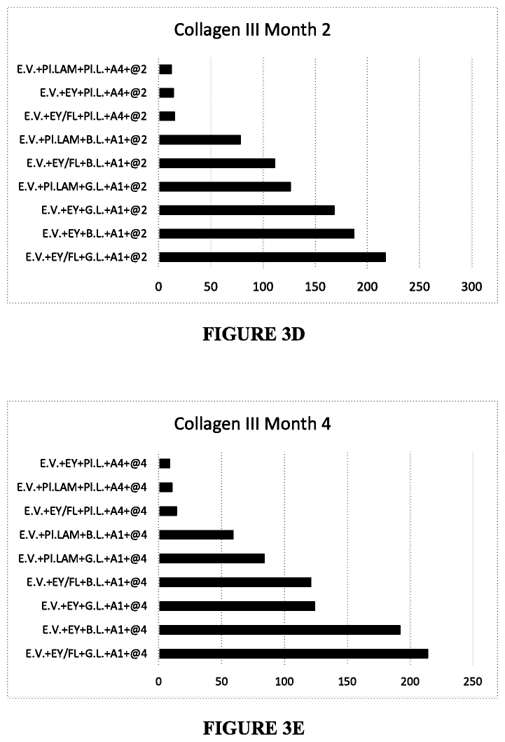

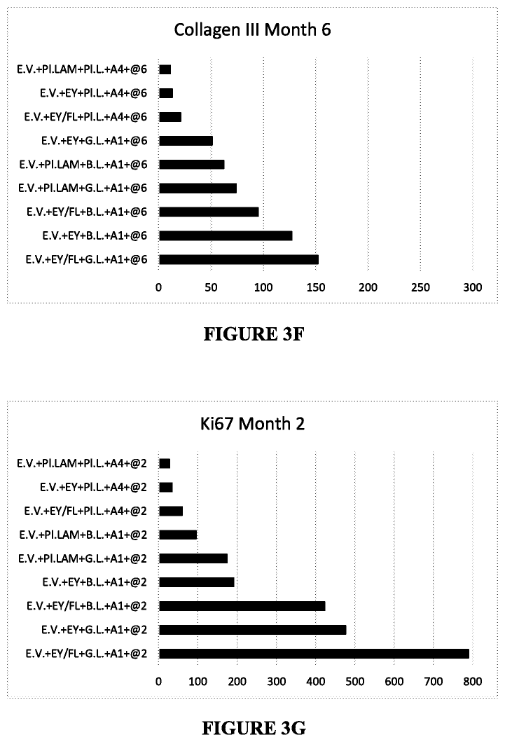

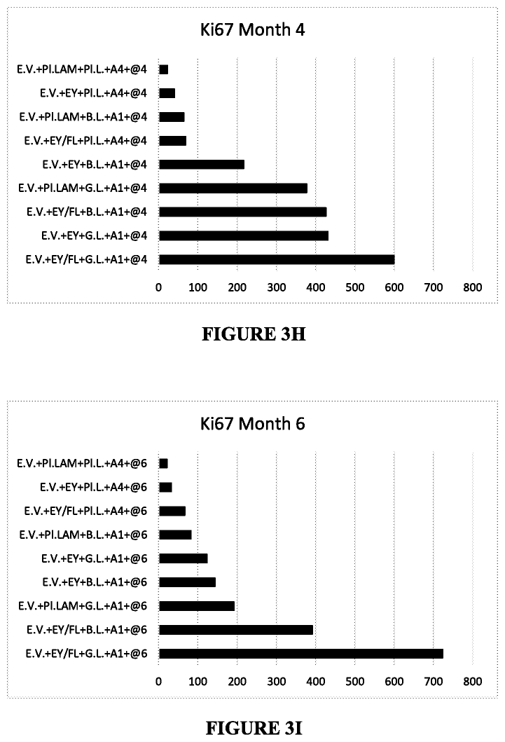

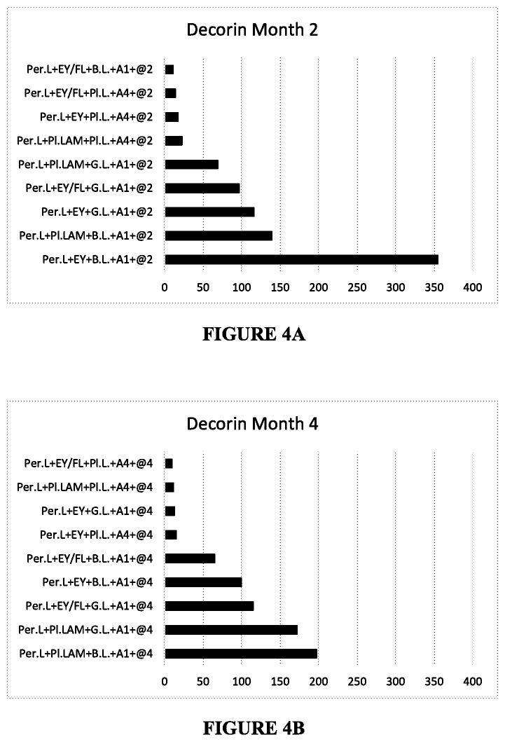

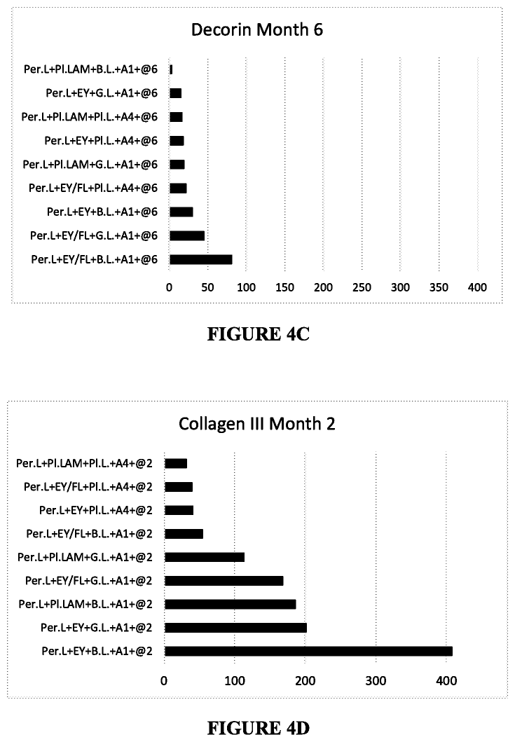

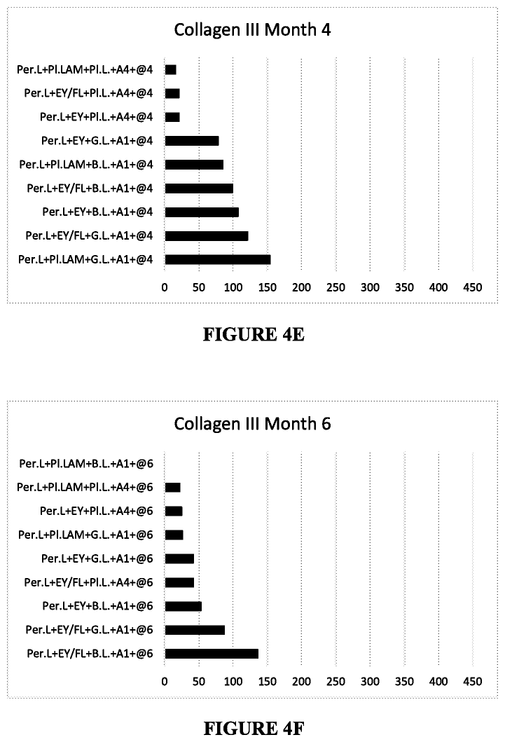

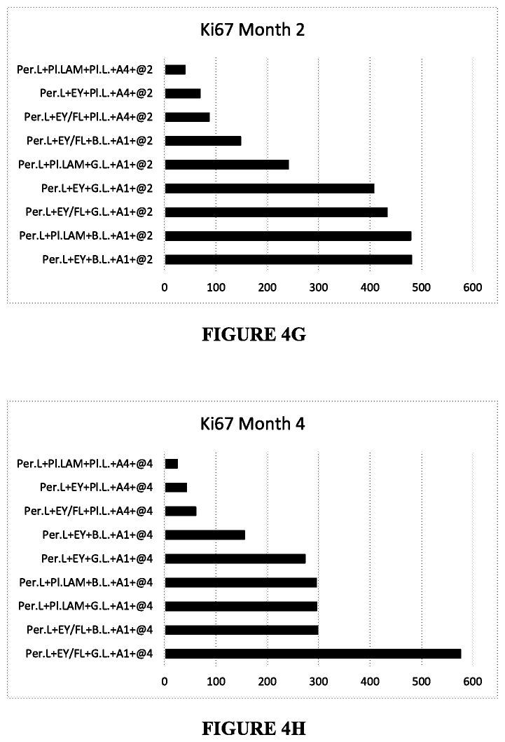

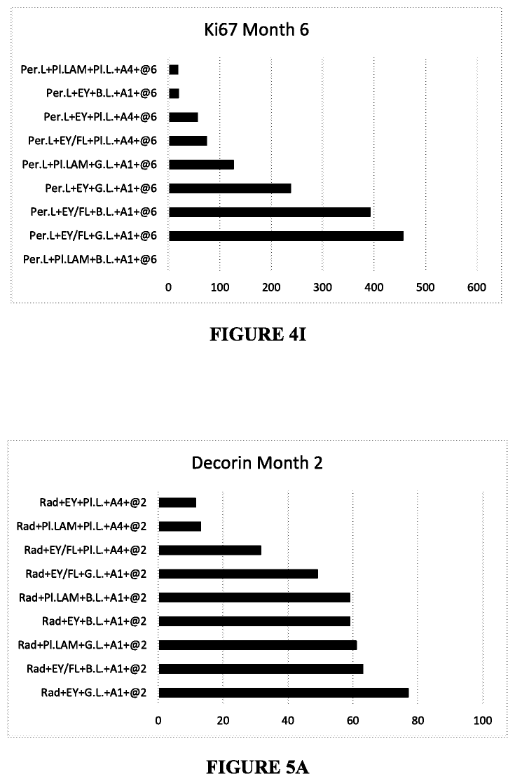

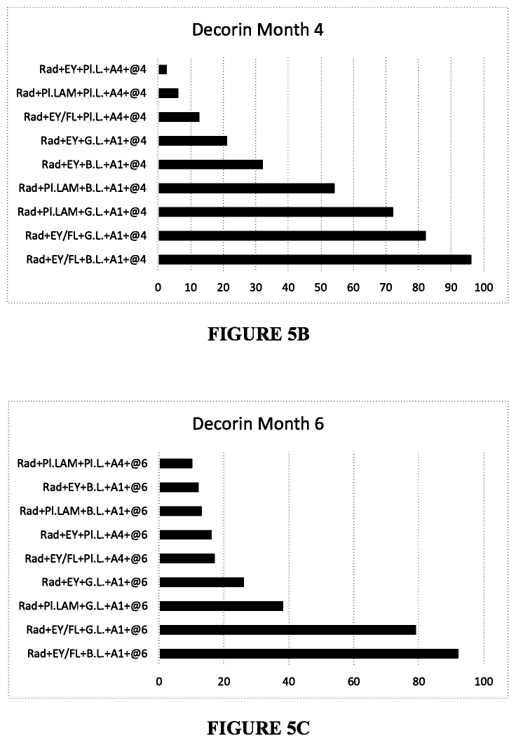

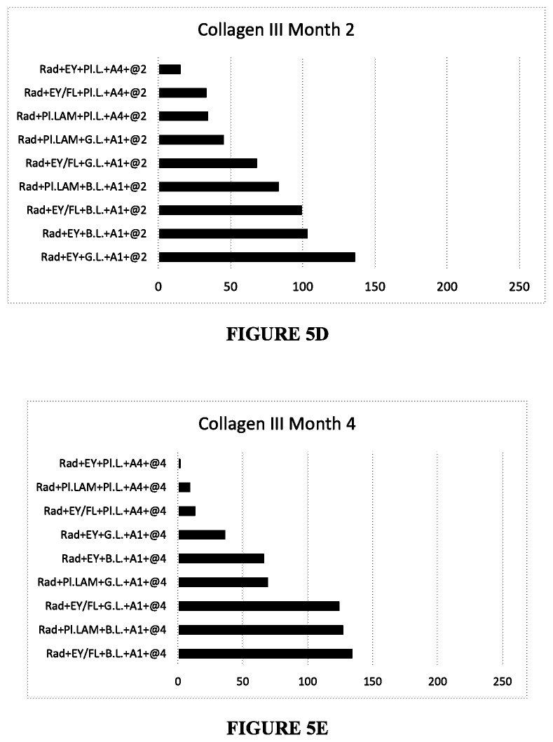

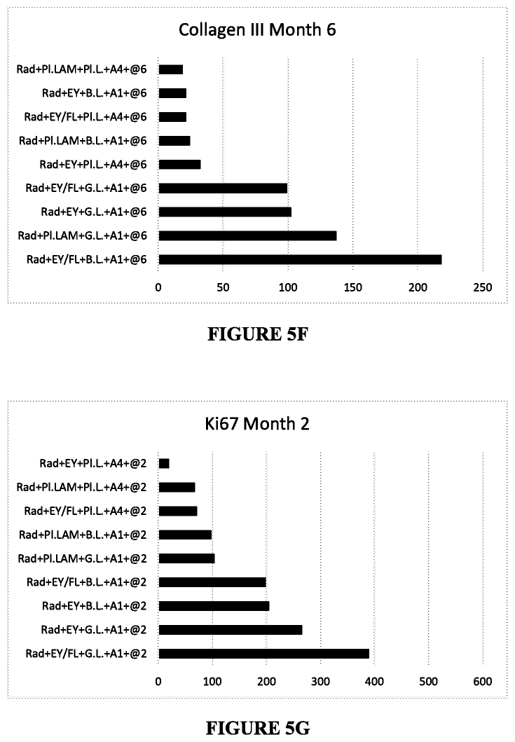

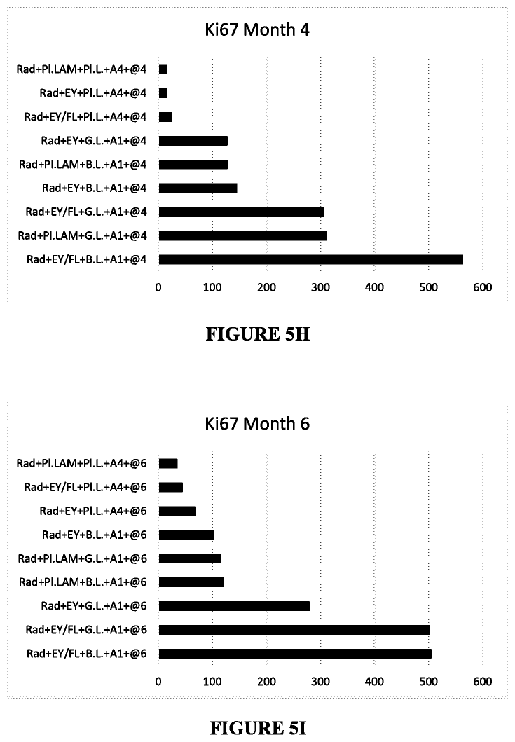

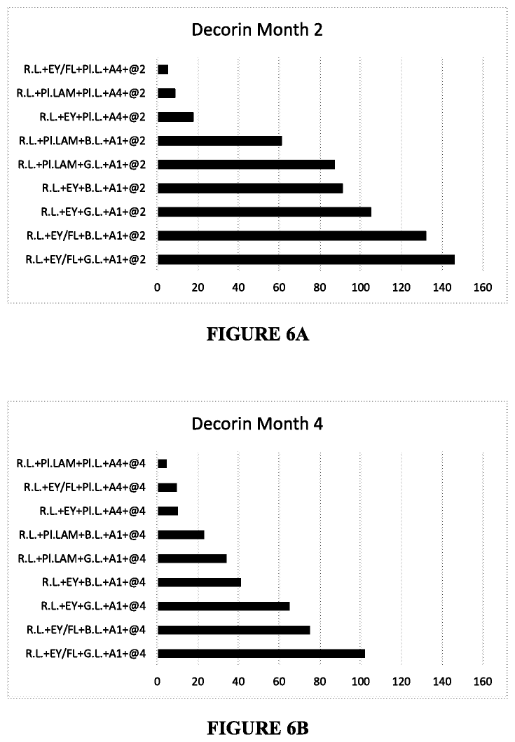

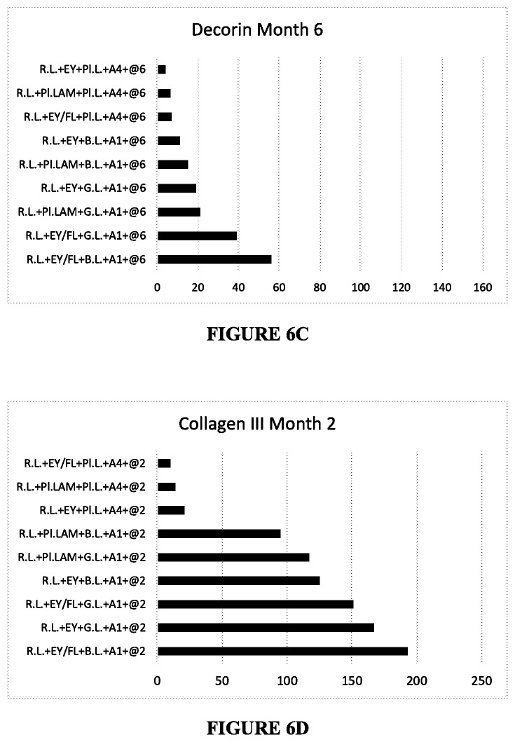

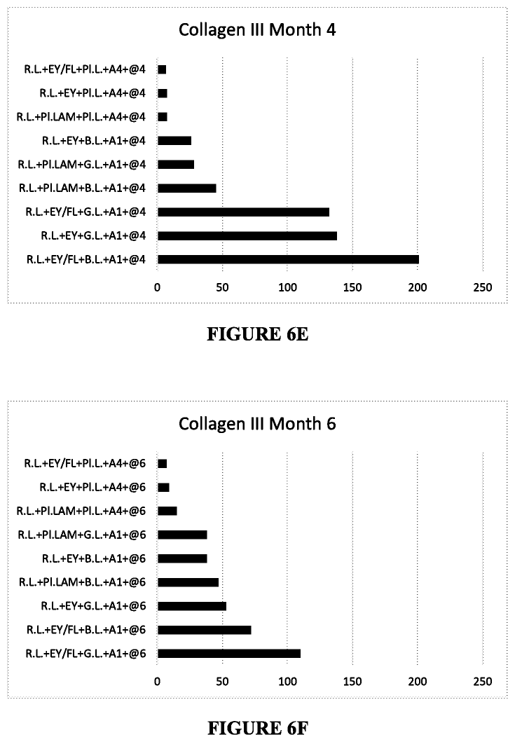

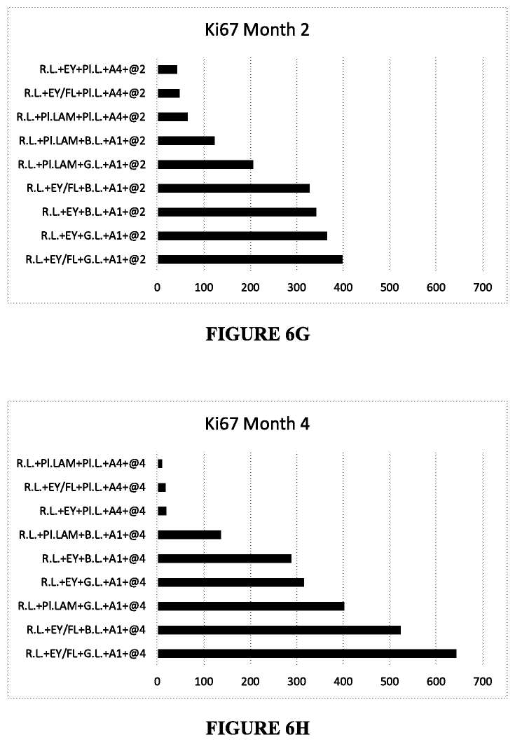

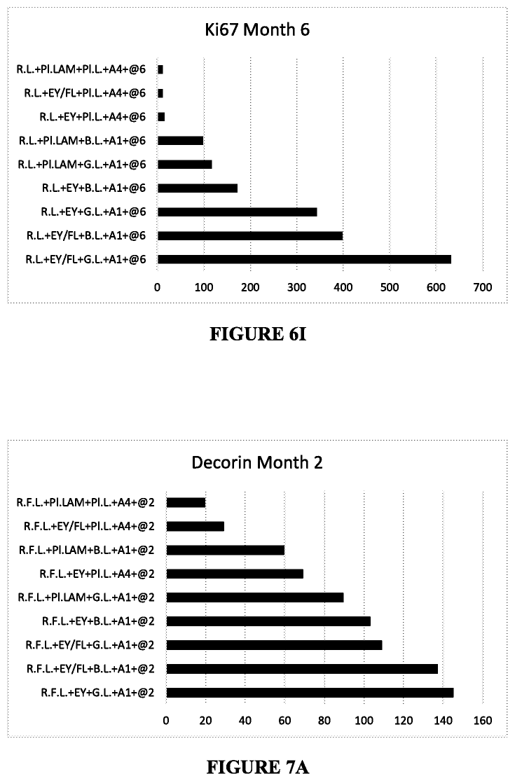

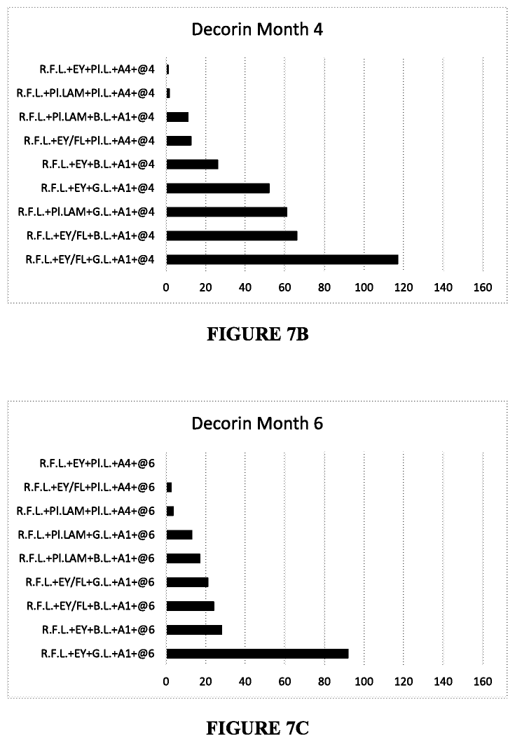

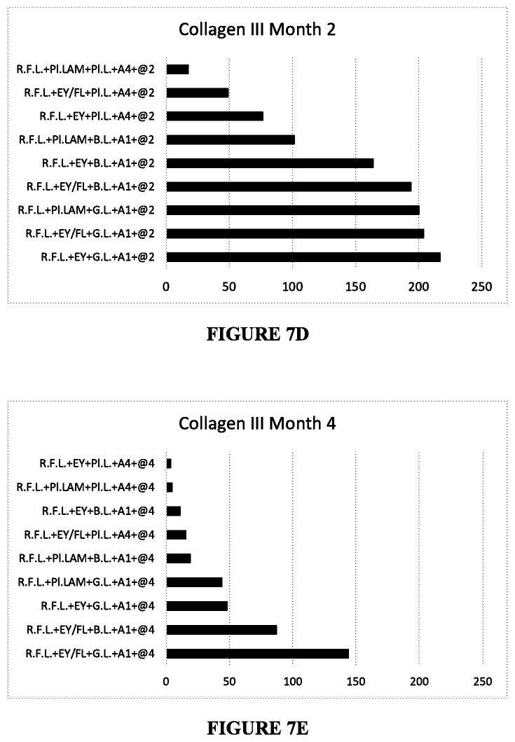

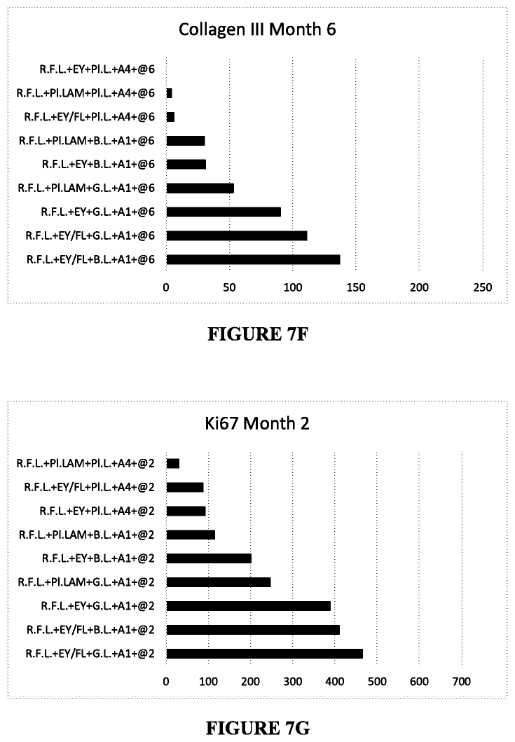

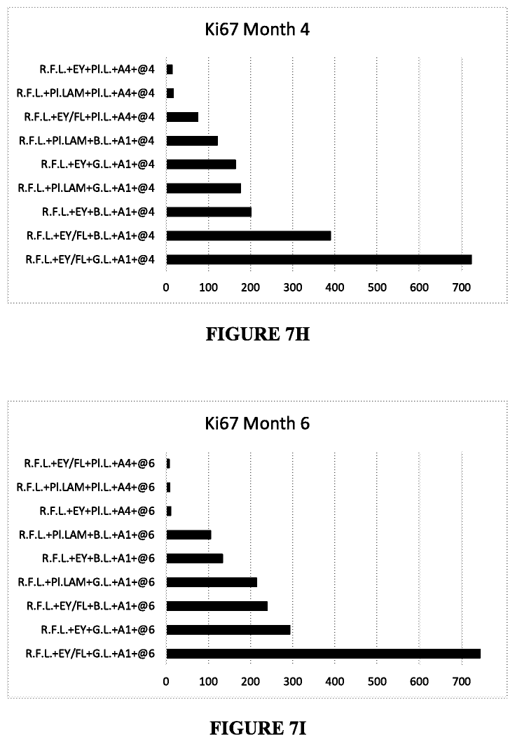

TABLE 4
Correspondence between results presented in FIGS. 2-7 and animals treated

| | Decorin | | | Collagen III | | | Ki67 | | |
|---|---|---|---|---|---|---|---|---|---|
| Dermal | 2 | 4 | 6 | 2 | 4 | 6 | 2 | 4 | 6 |
| Filler | months | months | months | months | months | months | months | months | months |
| Emerval ® | FIG. 2A | FIG. 2B | FIG. 2C | FIG. 2D | FIG. 2E | FIG. 2F | FIG. 2G | FIG. 2H | FIG. 2I |
| Classic | | | | | | | | | |
| Emervel ® | FIG. 3A | FIG. 3B | FIG. 3C | FIG. 3D | FIG. 3E | FIG. 3F | FIG. 3G | FIG. 3H | FIG. 3I |
| Volume | | | | | | | | | |
| Perlane ® | FIG. 4A | FIG. 4B | FIG. 4C | FIG. 4D | FIG. 4E | FIG. 4F | FIG. 4G | FIG. 4H | FIG. 4I |
| Radiesse ® | FIG. 5A | FIG. 5B | FIG. 5C | FIG. 5D | FIG. 5E | FIG. 5F | FIG. 5G | FIG. 5H | FIG. 5I |
| Restylane ® | FIG. 6A | FIG. 6B | FIG. 6C | FIG. 6D | FIG. 6E | FIG. 6F | FIG. 6G | FIG. 6H | FIG. 6I |
| Restylane ® | FIG. 7A | FIG. 7B | FIG. 7C | FIG. 7D | FIG. 7E | FIG. 7F | FIG. 7G | FIG. 7H | FIG. 7I |
| Fine Line | | | | | | | | | |

The data presented in FIGS. 2A-2I demonstrates that skin sections from animal models injected with PBM compositions comprising Eosin Y or Eosin Y/Fluorescein as light-absorbing molecule as well as with Emervel Classic® as tissue filler and exposed to blue or green light showed increase synthesis of one or more of Decorin, Collagen III and Ki67, at 2, 4 and 6 months after the phototreatment.

The data presented in FIGS. 3A-3I demonstrates that skin sections from animal models injected with PBM compositions comprising Eosin Y or Eosin Y/Fluorescein as light-absorbing molecule as well as with Emervel Volume® as tissue filler and exposed to blue or green light showed increase synthesis of one or more of Decorin, Collagen III and Ki67, at 2, 4 and 6 months after the phototreatment.

The data presented in FIGS. 4A-4I demonstrates that skin sections animal models injected with PBM compositions comprising Eosin Y or Eosin Y/Fluorescein as light-absorbing molecule as well as with Perlane® as tissue filler and exposed to blue or green light showed increase synthesis of one or more of Decorin, Collagen III and Ki67, at 2, 4 and 6 months after the phototreatment.

The data presented in FIGS. 5A-5I demonstrates that skin sections from animal models injected with PBM compositions comprising Eosin Y or Eosin Y/Fluorescein as light-absorbing molecule as well as with Radiesse® as tissue filler and exposed to blue or green light showed increase synthesis of one or more of Decorin, Collagen III and Ki67, at 2, 4 and 6 months after the phototreatment.

The data presented in FIGS. 6A-6I demonstrates that skin sections from animal models injected with PBM compositions comprising Eosin Y or Eosin Y/Fluorescein as light-absorbing molecule as well as with Restylane® as tissue filler and exposed to blue or green light showed increase synthesis of one or more of Decorin, Collagen III and Ki67, at 2, 4 and 6 months after the phototreatment.

The data presented in FIGS. 7A-7I demonstrates that skin sections from animal models injected with PBM compositions comprising Eosin Y or Eosin Y/Fluorescein as light-absorbing molecule as well as with Restylane Fine Line® as tissue filler and exposed to blue or green light showed increase synthesis of one or more of Decorin, Collagen III and Ki67, at 2, 4 and 6 months after the phototreatment.

Overall, these data suggest that photoactivated compositions comprising a light-absorbing molecule together with a dermal filler biomodulate collagen synthesis.

These data also demonstrate that the tissue filler alone (in absence of light-absorbing molecule) when illuminated by either blue or green light was capable of achieving biomodulation of collagen synthesis when illuminated by either blue or green light, suggesting that cross-linked molecules are able to perform biomodulation of collagen synthesis upon phototreatment.

### Example 3

### Assessing the Photo-Biomodulatory Effects of PBM Compositions on Collagen Production

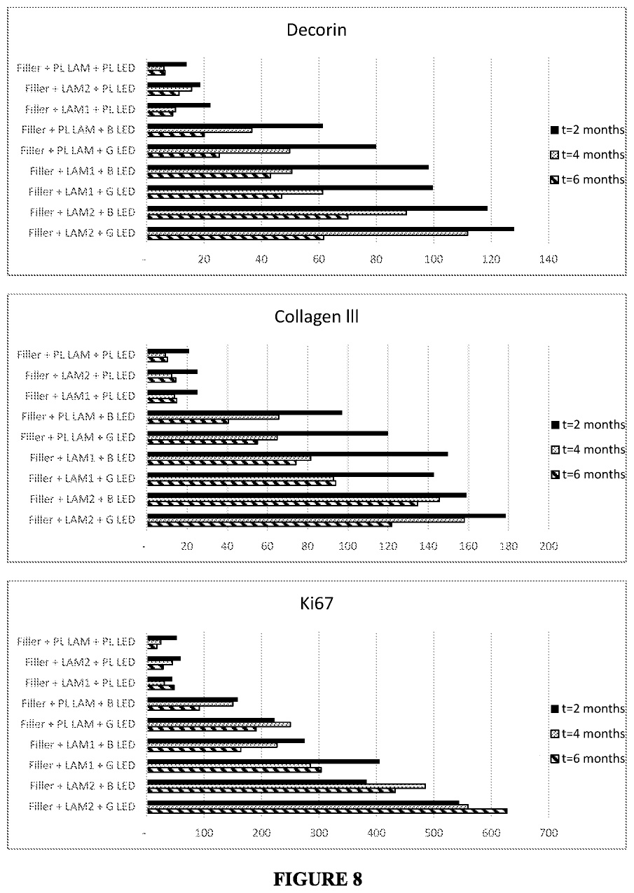

To assess the overall effect of the light-absorbing molecule on photo-biomodulation of collagen production, the average expression of collagen markers was calculated for each type of light accepting molecule (i.e., Eosin Y, Eosin Y+Fluorescein) and for each type of light (i.e., Blue light or Green light). The average data is presented in Table 5 and in FIG. 8.

TABLE 5
Overall averages of POS (cells/field)

| | 2 months post-illumination | | | 4 months post-illumination | | | 6 months-post-illumination | | |
|---|---|---|---|---|---|---|---|---|---|
| PBM composition and | | Collagen | | | Collagen | | | Collagen | |
| photo-treatment | Decorin | III | Ki67 | Decorin | III | Ki67 | Decorin | III | Ki67 |
| Filler + LAM2 + G LED | 128 | 178 | 544 | 112 | 158 | 559 | 62 | 122 | 627 |
| Filler + LAM2 + B LED | 119 | 159 | 383 | 90 | 145 | 485 | 70 | 135 | 433 |
| Filler + LAM1 + G LED | 100 | 143 | 405 | 61 | 93 | 285 | 47 | 94 | 304 |
| Filler + LAM1 + B LED | 98 | 150 | 275 | 51 | 82 | 227 | 43 | 74 | 164 |
| Filler + PL LAM + G LED | 80 | 120 | 222 | 50 | 65 | 250 | 25 | 55 | 190 |
| Filler + PL LAM + B LED | 61 | 97 | 158 | 37 | 66 | 150 | 20 | 41 | 92 |
| Filler + LAM1 + PL LED | 22 | 25 | 44 | 10 | 14 | 30 | 9 | 15 | 48 |
| Filler + LAM2 + PL LED | 19 | 25 | 59 | 16 | 12 | 45 | 11 | 14 | 29 |
| Filler + PL LAM + PL LED | 14 | 21 | 52 | 6 | 9 | 24 | 6 | 10 | 18 |

Filler = average of all dermal filler tested
LAM1 = Light-absorbing molecule 1 (Eosin Y)
LAM2 = Light-absorbing molecule 2 (Eosin Y + Fluorescein)
PL LAM = Placebo LAM
G LED = Green LED
B LED = Blue LED
PL LED = Placebo LED (no light/dark)

The data presented in Table 5 and in FIG. 8 shows the effect of varying the type of light-absorbing molecules and varying the type of light on stimulation of collagen synthesis markers. The data obtained demonstrates that skin sections injected with the PBM compositions comprising any one of the dermal filler identified in Table 5 and comprising Eosin Y or Eosin Y/Fluorescein as light-absorbing molecule(s) and exposed to blue or green light showed increase synthesis of one or more of Decorin (Panel A), Collagen III (Panel B) and Ki67 (Panel C), at 2, 4 and 6 months after the phototreatment.

### Example 4

### Assessing the Photo-Biomodulatory Effects of Tissue Fillers on Collagen Production

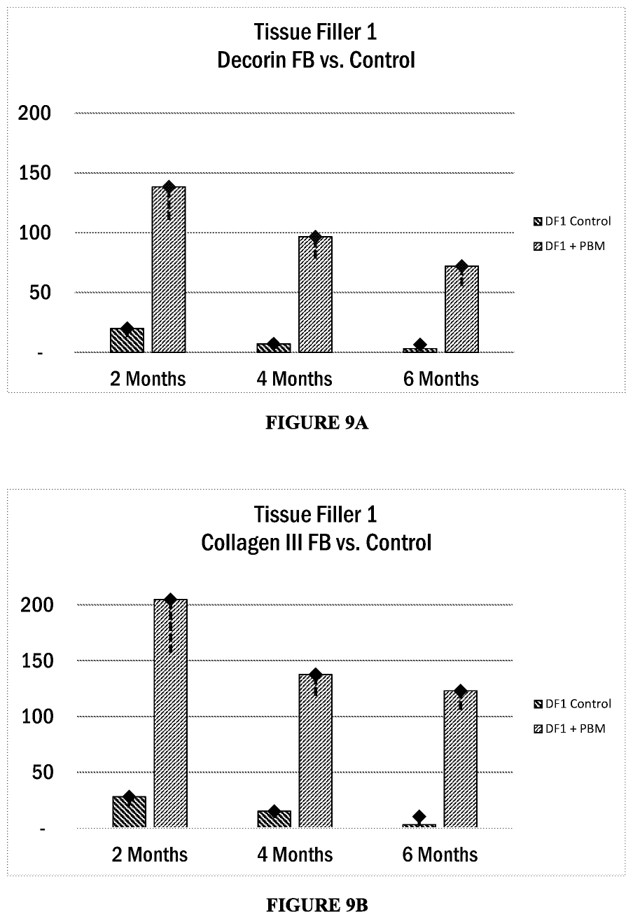

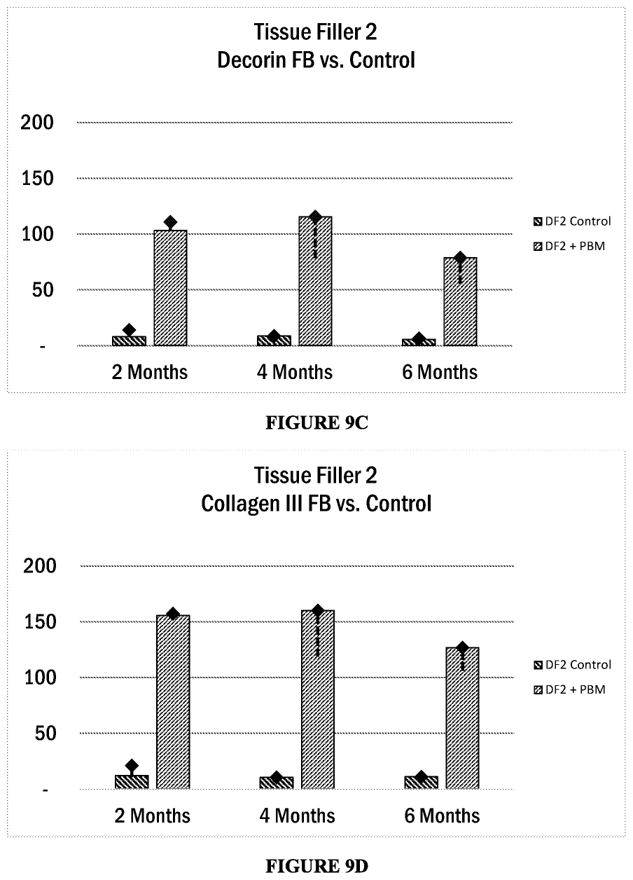

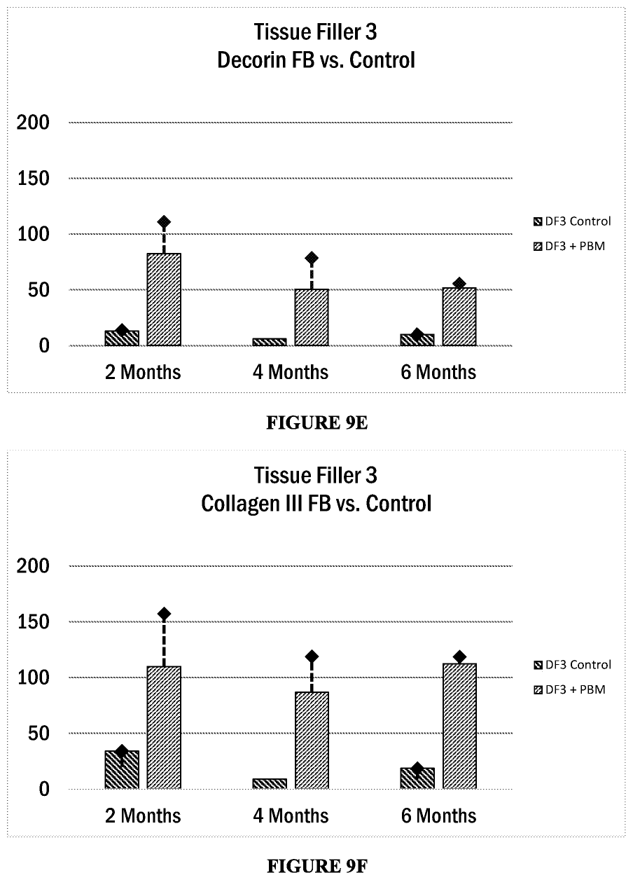

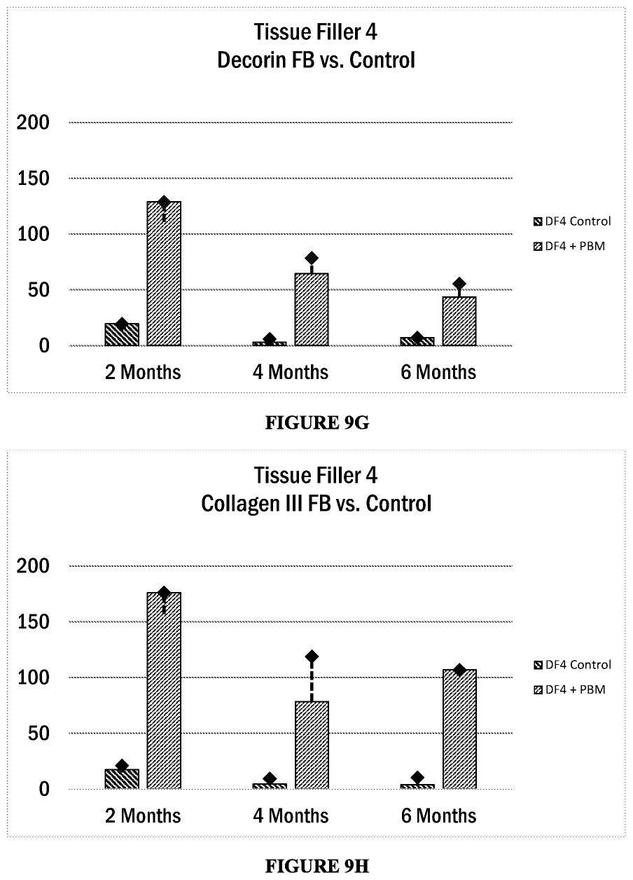

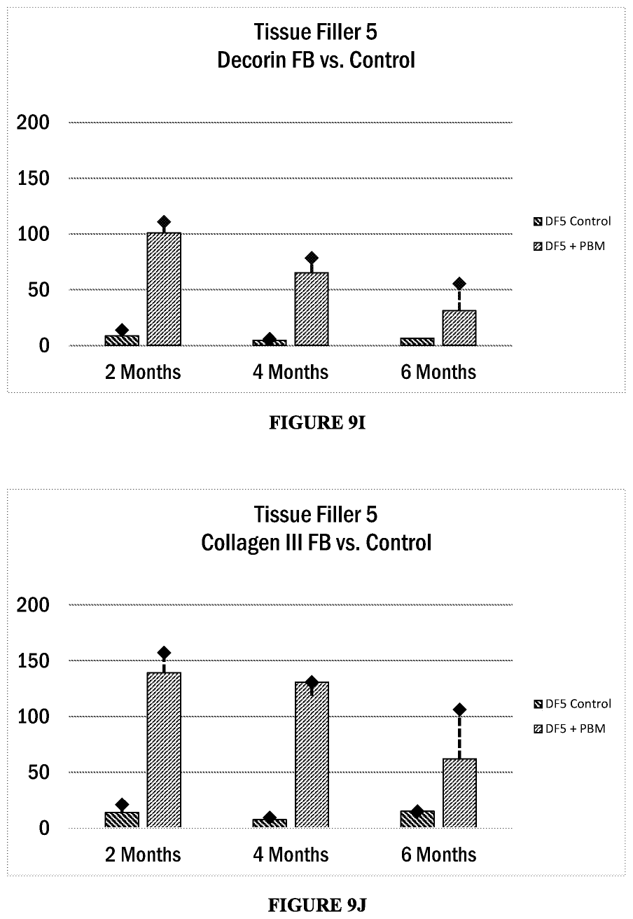

To assess the photo-biomodulatory effects of various types of tissue fillers (i.e., Emervel® Classic, Emervel® Volume, Perlane®, Radiesse®, Restylane®, Restylane® Fine Line) on collagen production the average expression of collagen markers was calculated for each type of tissue filler injected. Expression of Decorin and Collagen III in skin samples injected with dermal filler alone and skin samples injected with PBM compositions comprising tissue filler was compared. The data is presented in FIGS. 9A to 9J.

FIGS. 9A and 9B show the average effects for Dermal filler 1 (Emervel® Classic) on expression of Decorin (FIG. 9A) and expression of Collagen III (FIG. 9B) at the indicated time after phototreatment. FIGS. 9C and 9D show the average effects for Dermal filler 2 (Emervel® Volume) on expression of Decorin (FIG. 9C) and expression of Collagen III (FIG. 9D) at the indicated time after phototreatment. FIGS. 9E and 9F show the average effects for Dermal filler 3 (Perlane®) on expression of Decorin (FIG. 9E) and expression of Collagen III (FIG. 9F) at the indicated time after phototreatment. FIGS. 9G and 9H show the average effects for Dermal filler 4 (Radiesse®) on expression of Decorin (FIG. 9G) and expression of Collagen III (FIG. 9H) at the indicated time after phototreatment. FIGS. 9I and 9J show the average effects for Dermal filler 5 (Restylane®) on expression of Decorin (FIG. 9I) and expression of Collagen III (FIG. 9G) at the indicated time after phototreatment.

The data presented in FIGS. 9A-9J shows that injection of a tissue filler alone (in absence of light-absorbing molecule) when illuminated by either blue or green light was capable of achieving biomodulation of collagen synthesis (left columns) at 2, 4 and 6 months after phototreatment, the biomodulation of collagen synthesis was markedly increased in skin samples injected with both the dermal filler and the PBM composition (right columns). This was observed for all dermal fillers tested (i.e., Emervel® Classic, Emervel® Volume, Perlane®, Radiesse®, Restylane®, Restylane® Fine Line).

### Example 5

### Assessing the Photo-Biomodulatory Effects of PBM Compositions on Collagen I Production Levels

The aim of the experiment was to assess whether PBM compositions according to the present technology were capable of modulating Collagen I production/expression in an anti-age performance when comprising an intradermal filler such as Emervelle classique®, Emervelle Voluma® and Radiesse®.

To assess the foregoing, skin samples were injected with the PBM compositions as outlined in Table 6 and were phototreated as outlined in Example 1. The photo-treated skin samples were then assessed for expression of Collagen I. To assess for Collagen I expression, the photo-treated skin samples were stained with specific monoclonal antibody (Anti-Collagen Type I antibody from BioPorto Diagnostics, a mouse monoclonal antibody to 5D8 to Type I Collagen, Cola1, COL1A1, and collagen type I alpha 1 chain (cat. number CSI 008-01-1) for detection of Collagen I). Positive cells were counted and scheduled as described in Examples 1-4 above.

The data presented in Table 6 shows the effect of varying the type of light-absorbing molecules, tissue filler and light on Collagen I production levels. The data obtained demonstrates that skin sections injected with the PBM compositions comprising any one of the dermal filler identified in Table 6 and comprising Eosin Y or Eosin Y/Fluorescein as light-absorbing molecule(s) and exposed to blue or green light showed increase synthesis of Collagen I after the phototreatment.

TABLE 6
Levels of Collagen I production in skin treated with PBM compositions

| | | Collagen I |
|---|---|---|
| Tissue Filler | PBM Composition and phototreatment | (POS (cells/field) |
| Emervelle | Emervelle Classique, Eosin, Blue Light | 324 |
| | Emervelle Classique, Eosin, Green Light | 412 |
| | Emervelle Classique, Eosin, No Light | 31 |
| | Emervelle Classique, Eosin/Fluorescein, Blue Light | 422 |
| | Emervelle Classique, Eosin/Fluorescein, Green Light | 568 |
| | Emervelle Classique, Eosin/Fluorescein, No Light | 25 |
| | Emervelle Classique, Eosin/Fluorescein, No Light | 37 |
| | Emervelle Classique, Placebo, Blue Light | 68 |
| | Emervelle Classique, Placebo, Green Light | 198 |
| | Emervelle Classique, Placebo, No Light | 51 |
| | Emervelle Volume, Eosin, Green Light | 584 |
| | Emervelle Volume, Eosin/Fluorescein, Green Light | 709 |
| | Emervelle Volume, Placebo, Green Light | 165 |
| | No Injection, Placebo, Green Light | 18 |
| | Placebo, Placebo, Blue Light | 29 |
| | Placebo, Placebo, No Light | 6 |
| Radiesse | Radiesse, Eosin, Blue Light | 553 |
| | Radiesse, Eosin, Green Light | 651 |
| | Radiesse, Eosin, No Light | 61 |
| | Radiesse, Eosin/Fluorescein, Blue Light | 704 |
| | Radiesse, Eosin/Fluorescein, Green Light | 1110 |
| | Radiesse, Eosin/Fluorescein, No Light | 43 |
| | Radiesse, Placebo, Blue Light | 112 |
| | Radiesse, Placebo, Green Light | 191 |
| | Radiesse, Placebo, No Light | 81 |

These results suggest that different physicochemical properties of HA-based fillers, associated with distinct manufacturing technologies, may influence the fluorescence emitted by the light-absorbing molecule to effect biomodulation of biological/biochemical processes. The results also suggest that photo-stimulated HA-based fillers have the ability to effect biomodulation of some biological/biochemical processes. The results presented herein further suggest that as the tissue filler is depleted from the site of injection (due to normal degradation and/or absorption by the surrounding tissues), the amount and/or the activity (e.g., emission of fluorescence and/or biomodulation) of the light-activating agent at the site of injection also decreases.

While the present technology has been described in connection with specific embodiments thereof, it will be understood that it is capable of further modifications and this application is intended to cover any variations, uses, or adaptations of the invention following, in general, the principles of the present technology and including such departures from the present disclosure as come within known or customary practice within the art to which the present technology pertains and as may be applied to the essential features hereinbefore set forth, and as follows in the scope of the appended claims.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A method for effecting photo-biomodulation in a tissue, the method comprising:
administering at least one tissue filler to the tissue; and
illuminating the tissue with a source of actinic light;
wherein the illumination of the tissue filler with actinic light effects biomodulation in the tissue.

2. The method as defined in claim 1, wherein the at least one tissue filler is hyaluronic acid.

3. The method as defined in claim 2, wherein the hyaluronic acid is a mixture of short chains hyaluronic acid and long chains hyaluronic acid.

4. The method as defined in claim 2 or 3, wherein the hyaluronic acid is cross-linked.

5. The method as defined in claim 4, wherein the cross-linked hyaluronic acid is randomly cross-linked hyaluronic acid.

6. The method as defined in claim 4 or 5, wherein the cross-linked hyaluronic acid is cross-linked by divinyl sulfones.

7. The method as defined in claim 4 or 5, wherein the cross-linked hyaluronic acid is cross-linked by diglycidyl ethers.

8. The method as defined in claim 4 or 5, wherein the cross-linked hyaluronic acid is cross-linked by bis-epoxides.

9. The method as defined in any one of claims 2 to 8, wherein the hyaluronic acid is in a particulate form.

10. The method as defined in claim 9, wherein the particulate form is a microparticulate form.

11. The method as defined in claim 10, wherein the microparticles have a size between about 300 microns and about 700 microns.

12. The method as defined in any one of claims 1 to 11, wherein the at least one tissue filler is transparent or translucent.

13. The method as defined in any one of claims 1 to 12, wherein the at least one tissue filler is substantially colorless.

14. The method as defined in any one of claims 1 to 13, the at least one tissue filler is administered in combination with at least one light-absorbing molecule.

15. The method as defined in claim 14, wherein the at least one light-absorbing molecule is a xanthene dye.

16. The method as defined in claim 15, wherein the xanthene dye is selected from the group consisting of Eosin B, Eosin Y, Eosin derivative, Erythrosine, Fluorescein, Phloxin B and Rose Bengal.

17. The method as defined in claim 14, wherein the at least one light-absorbing molecule is Eosin Y and Fluorescein.

18. The method as defined in any one of claims 14 to 16, wherein the at least one light-absorbing molecule is dispersed between chains of the tissue filler.

19. The method as defined in any one of claims 1 to 18, wherein the step of administering is by injection.

20. The method as defined in claim 19, wherein the injection a subcutaneous injection.

21. The method as defined in claim 19, wherein the injection a subdermal injection.

22. The method as defined in claim 19, wherein the injection a dermal injection.

23. Use of at least one tissue filler to effect photo-biomodulation of a tissue, wherein illumination of the tissue filler with a source of actinic light effects biomodulation in the tissue.

24. Use of at least one photo-biomodulation composition to effect photo-biomodulation of a tissue, wherein the photo-biomodulation composition comprises at least one light-accepting molecule and at least one tissue filler; and wherein illumination of the photo-biomodulation composition with a source of actinic light effects biomodulation in the tissue.

25. A system for effecting photo-biomodulation in a tissue, the system comprising:
a photo-biomodulation composition in a form suitable for administration into the tissue; wherein the photo-biomodulation composition comprises at least one light-absorbing molecule and at least one tissue filler; and
a source of actinic light;
wherein illumination of the photo-biomodulation composition injected into the tissue with actinic light emitted from the source of actinic light triggers biomodulation of the tissue by the tissue filler.

26. Use of a photo-biomodulation composition comprising at least one tissue filler and at least one light-absorbing molecule in a method for modulating collagen synthesis in a tissue, wherein the method comprises a step of administration of the photo-biomodulation composition to an area to be treated within the tissue, and a step illumination of the area with light having a wavelength which can stimulate the at least one tissue filler and which can be absorbed by the at least one light-absorbing molecule; and wherein the method biomodulates collagen synthesis in the area.

27. Use of a photo-biomodulation composition comprising at least one tissue filler and at least one light-absorbing molecule in a method for management of a wound, wherein the method comprises a step of administration of the photo-biomodulation composition to an area of skin comprising the wound, and a step illumination of the area with light having a wavelength which can stimulate the at least one tissue filler and which can be absorbed by the at least one light-absorbing molecule; and wherein the method biomodulates collagen synthesis in the area.

28. A tissue filler composition comprising:
at least one tissue filler; and
at least one light-absorbing molecule;
wherein the composition substantially colourless and is suitable for injection into a tissue.

29. In a combination, at least one tissue filler in a form suitable for administration into a tissue, and a source of actinic light, the source of actinic light for stimulating the at least one tissue filler, wherein stimulation of the at least one tissue filler by actinic light emitted by the source of actinic light triggers biomodulation in the tissue.

30. In a combination, at least one tissue filler in a form suitable for administration into a tissue, at least one light-absorbing molecule, and a source of actinic light, the source of actinic light for stimulating the at least one tissue filler and activating the at least one light-absorbing molecule, wherein stimulation of the at least one tissue filler and activation of the at least one light-absorbing molecule by actinic light emitted by the source of actinic light triggers biomodulation in the tissue.

31. In a combination, at least one tissue filler in a form suitable for administration into a tissue, and at least one light-absorbing molecule in a form suitable for administration in to a tissue, wherein stimulation of the at least one tissue filler and activation of the at least one light-absorbing molecule by light triggers biomodulation in the tissue.

32. A method for minimizing appearance of fine lines on skin, wherein the method comprises:
administering at least one tissue filler into the fine lines; and
illuminating the fine lines with a source of actinic light;
wherein the illumination of the fine lines with actinic light minimizes the appearances of the fine lines.

33. A method for minimizing appearance of fine lines on skin, wherein the method comprises:
administering at least one tissue filler and at least one light-absorbing molecule into the fine lines; and
illuminating the fine lines with a source of actinic light;
wherein the illumination of the fine lines with actinic light minimizes the appearances of the fine lines.

34. The method as defined in claim 32 or 33, wherein the fine lines are located around an upper lip area of a human.

35. A tissue filler composition, wherein the tissue filler composition is a cross-linked tissue filler network capable of retaining and transferring energy upon being stimulated by actinic light emitted by a light source.

36. A tissue filler composition, wherein the tissue filler composition is a cross-linked tissue filler network capable of retaining and transferring energy to one or more light-absorbing molecules upon being stimulated by actinic light emitted by a light source.

37. A method for effecting photo-biomodulation in a tissue, the method comprising:
administering a first photo-biomodulation composition into an area of a tissue, wherein the photobiomodulation composition comprises at least one light-absorbing molecule and at least one tissue filler;
topically applying a second photo-biomodulation composition to the area of the tissue; and
illuminating the area of the tissue with a source of actinic light;
wherein the illumination of the area of the tissue with actinic light effects biomodulation in the tissue.
